SEQUENCE LISTING (12 sequences)

SEQ ID NO: 1 - DNA, 29 bp, Artificial Sequence
  primer ODH68
cgggatccagaccaaccactcgaactact

SEQ ID NO: 2 - DNA, 29 bp, Artificial Sequence
  primer ODH69
cgggatccgaaggtgaaccaccatatttg

SEQ ID NO: 3 - DNA, 29 bp, Artificial Sequence
  primer OEL109
gaagatcttgtaatcaatgttcccgtgga

SEQ ID NO: 4 - DNA, 29 bp, Artificial Sequence
  primer OEL110
gaagatctcatcgtgatattaaatccgat

SEQ ID NO: 5 - DNA, 4492 bp, Candida albicans
  CDS (355)...(4044)
gtacccactttacaatcacttacaagtcaaataattacaacttgacaatcctcactttaagtctaacgtatatacgcgtacaccatcttatactccacatacatattggattcaatttttattttattgtttagtttatatccaaccactgacaattaccaatagttttcaattaatattcacaatttaactatttgtttgacagctgaaaagagataaaaaaagaatcaagtgctataactcacaagggctagaaataagtttgcaaaaaacaagttttaaaaatagtaactgcacttttgttgactctttcacctccccattgaatttaactgaacacaaataaagcctatcatgagcatactttcagagaacaatcctacaccaacatcaataacagatccaaatgagtcttctcatctacacaacccagagttaaactctggaacgagggttgcttctggacctggacctggacctgaagttgaatcaacaccactagcacccccaactgaggtcatgaatactacatcagctaatacttcttcattaagtttagggtctccaatgcacgagaaaataaaacaatttgatcaagacgaggttgacactggggaaactaatgataggactatagaatctggatctagtgatattgatgattcacaacaatcacataacaacaacaacaacaacaacaacaacaacaacgagagcaatccagaatcaagtgaaggcgatgatgaaaaaacccaaggaatgcctcctcgaatgccagggacattcaatgtgaaaggtttgcaccaaggggatgatagtgacaatgaaaaacagtacaccgagctaactaaatcaatcaataaacgtaccagtaaagattcgtattctcctggcacacttgaaagtcccggtactcttaatgcattggaaacaaataatgtctcaccagcagttatagaggaagaacaacatacactgtctttggaagatttgtcattgtccttacaacaccaaaatgaaaatgcaagattatctgcaccccgcagtgcaccgccacaggttccgacttcaaagacatcgtcatttcacgatatgagtctggttatatcttcatcaacttctgtgcataagataccatcaaatccaacttcaactcgaggttctcatttatcaagttacaaatctacattggaccctgggaaacctgcacaagcagcagcaccaccaccaccagaaatagacattgacaatttattaaccaaaagtgaattggatctggaaacagacacattgagtagtgccacaaattctccaaaccttttaagaaatgatactttacaaggaattccaacaagagatgacgaaaatattgatgacctgccccgtcaactatcacaaaatactagtgcgacgtcaagaaatacttcgggaacatcgacttctacagtggtgaaaaattcaagatctggtacgtcaaaatcaacctcaacctcaactgctcataaccaaacagcagcaattactcctataatcccgagtcacaacaagtttcatcaacaagtgataaataccaatgcaacaaatagttcatcttcactagaaccattgggggttggcataaattcaaatctgtctcctaaaagtgggaaaaagcggaaaagtggaagtaaagtccgaggtgtgttttcgtcaatgtttgggaaaaacaagtcaacgtcatcatcgtcgtcttcaaactcaggtctgaatagccactcacaggaagtcaatattaagatcagtactccattcaatgccaagcaccttgcccatgtgggcattgatgataatggttcatacaccggtttgccaatagagtgggaaagattattatctgctagtggtattaccaagaaggaacaacaacagcacccacaagcagtgatggatatagtggcgttttatcaagatacaagtgaaaaccctgatgacgctgcatttaaaaagtttcattttgataataataaaagtagttcgagtggttggtctaatgaaaatactccaccagcaacaccgggtgggagtaacagtggcagtggcagtggtggcggtggcgctccttcaagtccccatcgtacacctccttcatcgatcattgaaaaaaacaacgttgaacaaaaagtgattaccccatctcagtcaatgccaacaaagacagagagtaaacagctggaaaaccagcacccacatgaagataatgctactcagtatacaccaagaacaccaacatcccatgtacaagagggtcaatttattccaagtagaccagctccgaaaccaccatcaacaccgctttcttccatgagtgtgtcacataaaacaccttcttcgcaatcattaccaaggagtgattcacaatccgatattcgttcttcaacccctaaatcacatcaagatgtttcgccaagcaagatcaaaattcgttcaatttcgtcaaaatcattaaagtcaatgcggtctagaaaaagtggggataagtttactcatattgcacctgctcctccaccaccatcattaccttcaattcctaaatcaaagtcgcattcggcatctttgtcaagtcaattgagaccagcaacaaatggatcaacaactgcccctattccagcaagtgccgcgtttggtggtgagaataatgctttaccaaaacaaagaataaatgagttcaaggctcatagagcacctccaccacctccactggcaccacctgcaccacctgtgcctcctgctccaccagccaatttattatcggaacagacttctgagatacctcaacaacgtactgctcctctgcaagcattagctgatgttactgccccaactaatatttatgaaattcaacaaactaaatatcaggaagcacaacagaaattacgtgagaagaaggctagagaacttgaagaaatacaaagactacgagagaagaatgaaagacaaaatagacaacaggagactgggcaaaataatgctgacacggctagcggtggtagtaatattgctccaccagtacctgtaccaaataaaaaaccgccttctggatctggtggtggccgtgatgccaaacaagcagctttgatagcccaaaagaaacgagaagaaaagaaacgtaaaaacttacaaattattgccaaattaaagacaatttgtaatcctggagatccaaatgaattatatgttgatttagttaaaattggtcaaggtgcctccggtggagttttccttgctcatgatgttcgtgataaatccaatattgttgccataaaacaaatgaatttagaacaacaacctaaaaaagaattaattattaatgaaattttggttatgaaaggtagtctgcatcccaatattgtcaattttattgattcatatcttttaaaaggtgatttatgggtgattatggaatatatggaaggtggatcccttaccgatatagtgactcatagtgttatgaccgaaggtcaaattggagttgtatgtcgtgaaactttgaaaggtcttaaatttttacattccaaaggggttatccatcgtgatattaaatccgataatattttattaaatatggatggtaacatcaagatcactgattttgggttttgtgctcaaatcaatgaaatcaatctgaaacgtatcactatggtgggtacaccatattggatggcaccagaaattgtttcacgtaaagagtatggtccaaaagttgatgtttggtcattaggtatcatgattatagaaatgttagaaggtgaaccaccatatttgaatgaaactccattgagggcattatatcttattgcaactaatggtacaccaaaattaaaagatcctgaatctttaagttatgatattagaaaatttttggcatggtgtttacaagttgactttaataaaagagctgatgctgatgaattattacatgataattttattactgaatgtgatgatgtatcgtcgttaagtccattagtgaaaattgctcgattgaaaaaaatgagtgaatctgattaatgaatggtggagttatcctagaaataaaaacatttaaaaaaaaagaagaagaacaacaagaaccctaaattctactgctgtcaatatattggctaatttccattctcgtttctatttctatttcgtttttattctttgaattattattgttagtggtagagatttttactagtatattttttttattcatttttatatttgtatttatatatatatttttcatttagtatttacttacactgcagtatctttcttttctgtgtagatgatatgtagtaataagttaacttgttcaagacagtgaatggaaatatattatagcttgactatataaggtggagagctgtaattggctttccgtatagaaaagtcttgaacaaacgttaccagatttctgctattcttatttggtacgattcgggcgtatgataggtttattgagctc

SEQ ID NO: 6 - protein, 1230 aa, Candida albicans
MSILSENNPTPTSITDPNESSHLHNPELNSGTRVASGPGPGPEVESTPLAPPTEVMNTTSANTSSLSLGSPMHEKIKQFDQDEVDTGETNDRTIESGSSDIDDSQQSHNNNNNNNNNNNESNPESSEGDDEKTQGMPPRMPGTFNVKGLHQGDDSDNEKQYTELTKSINKRTSKDSYSPGTLESPGTLNALETNNVSPAVIEEEQHTLSLEDLSLSLQHQNENARLSAPRSAPPQVPTSKTSSFHDMSLVISSSTSVHKIPSNPTSTRGSHLSSYKSTLDPGKPAQAAAPPPPEIDIDNLLTKSELDLETDTLSSATNSPNLLRNDTLQGIPTRDDENIDDLPRQLSQNTSATSRNTSGTSTSTVVKNSRSGTSKSTSTSTAHNQTAAITPIIPSHNKFHQQVINTNATNSSSSLEPLGVGINSNLSPKSGKKRKSGSKVRGVFSSMFGKNKSTSSSSSSNSGLNSHSQEVNIKISTPFNAKHLAHVGIDDNGSYTGLPIEWERLLSASGITKKEQQQHPQAVMDIVAFYQDTSENPDDAAFKKFHFDNNKSSSSGWSNENTPPATPGGSNSGSGSGGGGAPSSPHRTPPSSIIEKNNVEQKVITPSQSMPTKTESKQLENQHPHEDNATQYTPRTPTSHVQEGQFIPSRPAPKPPSTPLSSMSVSHKTPSSQSLPRSDSQSDIRSSTPKSHQDVSPSKIKIRSISSKSLKSMRSRKSGDKFTHIAPAPPPPSLPSIPKSKSHSASLSSQLRPATNGSTTAPIPASAAFGGENNALPKQRINEFKAHRAPPPPPLAPPAPPVPPAPPANLLSEQTSEIPQQRTAPLQALADVTAPTNIYEIQQTKYQEAQQKLREKKARELEEIQRLREKNERQNRQQETGQNNADTASGGSNIAPPVPVPNKKPPSGSGGGRDAKQAALIAQKKREEKKRKNLQIIAKLKTICNPGDPNELYVDLVKIGQGASGGVFLAHDVRDKSNIVAIKQMNLEQQPKKELIINEILVMKGSLHPNIVNFIDSYLLKGDLWVIMEYMEGGSLTDIVTHSVMTEGQIGVVCRETLKGLKFLHSKGVIHRDIKSDNILLNMDGNIKITDFGFCAQINEINLKRITMVGTPYWMAPEIVSRKEYGPKVDVWSLGIMIIEMLEGEPPYLNETPLRALYLIATNGTPKLKDPESLSYDIRKFLAWCLQVDFNKRADADELLHDNFITECDDVSSLSPLVKIARLKKMSESD

SEQ ID NO: 7 - DNA, 3496 bp, Candida albicans
  CDS (432)...(3344)
gaattctttttagaagagaaagaaaaaattcccaaaaaaaaaagatttcatttaattccacgggaacattgattacaaccacgtcaacagtttcccttttatattgaaatcaacattcaattttgtctttttttttttttcattgatttttccccaatctttttatcttcatattaatattggatatcaattactaatactgtcagggatagtttagtaaatatttacattctccattcaatcctaaatttttttttatatagctagtttttggttgaaaaaaaaaaaatagggggaaggaagtttttttttctatttatttaattgttttgattccaaccatattgtatatttgtcttgtcagttatattactttcttgttacttaattattaattatttgctatattattgaattgaatcctcaaaagaatgacaagtatttatacatcagatttgaaaaaccatagacgtgcgccacctccaccaaatggggcagctggctctggctcaggttctggctcaggttctggttctggttctggcagtttggctaatattgttaccagttctaatagtcttggcgtaacagcaaatcaaaccaaacctattcaattaaatataaattctagcaaacgtcaatcaggttgggttcatgttaaagatgatggtattttcacatcatttagatggaacaaacggtttatggttattaatgataaaactttaaacttttataaacaagaaccatattctagtgatggtaattccaattctaatacccctgatttatcattcccactatatttaattaataatattaatttgaaaccaaactccgggtatagcaaaacttcacaatcatttgaaattgttcccaaaaacaataataaatcaattttgatttctgttaaaaccaataatgattatttggattggctagatgcattcaccacaaaatgtcctttagtacaaattggtgaaaataatagtggtgtatcaagtagtcaccctcatttacaaattcaacatttaaccaatggttccttgaacggcaactcatcttcatcaccaacatctggattattatcttcttcagtgctaactggaggtaattctggcgtttctggtcctattaatttcactcataaagtacacgtgggatttgatcctgccagtggtaattttactggattaccagacacttggaaaagtttattacaacattcgaaaatcactaatgaggattggaaaaaagatcctgttgctgttattgaagttttagaattttattccgatataaatggaggtaattcagctgctggaactccaattggatcacccatgatcaattccaaaaccaacaataataataatgaccctaacaattactcatcaaccaaaaacaatgtccaagaggcaaatttacaagaatgggtaaaacctccagcaaaatctactgtctcacaattcaaacctagtcgagctgcaccaaaaccaccaactccatatcatttgacacaactaaatggctcttcccaccaacatacatcatcatcaggctcattacctagttctggtaataataataataataacagcactaacaataataatactaaaaacgtttcaccattgaataatttgatgaataaatctgaacttattcctgctagaagagctccaccacctccaacaagtggcacatcttcagatacatattctaataagaatcatcaagatagatctggatatgaacaacaacgtcaacaacgtactgactcatcacaacaacaacaacaacaaaagcaacatcaatatcaacagaaatcccaacaacaacaacaacaaccacaacaaccattatctctgcatcaaggtgggacttcgcatattccgaaacaagtacctcctacattaccatcatctggaccacccactcaggctgcttcaggaaaatcaatgccatctaaaattcatcctgatcttaagattcaacaaggcacaaataattatattaagagtagcggtactgatgctaatcaagtcgatggtgatgctaaacaatttattaaaccatttaatttacaactgaaaaagagtcagcaacaattggcatcaaaacaaccgtcaccaccttcatctcaacaacagcaacaaaaacctatgacatcacatggattaatgggtacatcacattcagttactaaaccattgaatccagtcaatgatccaatcaaaccattaaatttgaaatcatctaaatccaaagaagcattaaatgaaactctgggggtgctgaaaacaccatcacctacagataaatcaaataaaccaactgcacctgctagtggtcctgcagtgaccaaaacagctaaacaactcaagaaggaacgagaaagattgaatgatttacaaatcattgctaaattgaaaacagtggttaataatcaagatcctaaaccattatttagaattgttgaaaaagctggtcaaggtgctagtgggaatgtttatttggcggaaatgatcaaagataataatcgaaagattgcgattaaacaaatggatcttgatgctcaaccccgtaaagagttaataataaatgaaatcttggttatgaaagatagtcaacataaaaatattgttaattttttggattcttatttaattggtgataatgaattatgggtaattatggaatatatgcaaggtggttcattaacggaaatcattgaaaataatgattttaaattgaatgaaaaacaaattgccactatatgttttgaaaccttaaaggggttacaacatttacataaaaaacatattattcatcgtgatattaaatccgataatgttttattagatgcatatggtaatgttaaaatcactgattttggattttgtgctaaattaactgatcaaagaaataaacgtgccacaatggtggggacaccatattggatggcacctgaagtggttaaacaaaaggaatatgatgaaaaagttgatgtttggtcattggggattatgactattgaaatgattgaaggagaaccaccttatttgaatgaagaaccattaaaagctttatatcttatagctactaatggtacaccaaaattgaaaaaacccgaattattatcgaattcaattaaaaaattcttatcaatttgtctttgtgttgatgttagatatcgtgctagtactgatgaattattagaacattcatttattcaacataaatcagggaaaattgaagaattggcaccattattagaatggaaaaaacaacaacaaaagcatcaacagcataaacaagaaacactggatacaggatttgcatagagattgaatatagccgtagaaaactggtactttggttttggtataatatatttatgtgatgtgttgtgtgtatggttagtttagatttggatttttagttttttagagtttagtttttcaatttttagttttagagacaatattctaga

SEQ ID NO: 8 - protein, 971 aa, Candida albicans
MTSIYTSDLKNHRRAPPPPNGAAGSGSGSGSGSGSGSGSLANIVTSSNSLGVTANQTKPIQLNINSSKRQSGWVHVKDDGIFTSFRWNKRFMVINDKTLNFYKQEPYSSDGNSNSNTPDLSFPLYLINNINLKPNSGYSKTSQSFEIVPKNNNKSILISVKTNNDYLDWLDAFTTKCPLVQIGENNSGVSSSHPHLQIQHLTNGSLNGNSSSSPTSGLLSSSVLTGGNSGVSGPINFTHKVHVGFDPASGNFTGLPDTWKSLLQHSKITNEDWKKDPVAVIEVLEFYSDINGGNSAAGTPIGSPMINSKTNNNNNDPNNYSSTKNNVQEANLQEWVKPPAKSTVSQFKPSRAAPKPPTPYHLTQLNGSSHQHTSSSGSLPSSGNNNNNNSTNNNNTKNVSPLNNLMNKSELIPARRAPPPPTSGTSSDTYSNKNHQDRSGYEQQRQQRTDSSQQQQQQKQHQYQQKSQQQQQQPQQPLSLHQGGTSHIPKQVPPTLPSSGPPTQAASGKSMPSKIHPDLKIQQGTNNYIKSSGTDANQVDGDAKQFIKPFNLQLKKSQQQLASKQPSPPSSQQQQQKPMTSHGLMGTSHSVTKPLNPVNDPIKPLNLKSSKSKEALNETLGVLKTPSPTDKSNKPTAPASGPAVTKTAKQLKKERERLNDLQIIAKLKTVVNNQDPKPLFRIVEKAGQGASGNVYLAEMIKDNNRKIAIKQMDLDAQPRKELIINEILVMKDSQHKNIVNFLDSYLIGDNELWVIMEYMQGGSLTEIIENNDFKLNEKQIATICFETLKGLQHLHKKHIIHRDIKSDNVLLDAYGNVKITDFGFCAKLTDQRNKRATMVGTPYWMAPEVVKQKEYDEKVDVWSLGIMTIEMIEGEPPYLNEEPLKALYLIATNGTPKLKKPELLSNSIKKFLSICLCVDVRYRASTDELLEHSFIQHKSGKIEELAPLLEWKKQQQKHQQHKQETLDTGFA

SEQ ID NO: 9 - DNA, 1031 bp, Candida albicans
  CDS (271)...(843)
caaccaaaccaactttcatccttctaccaatatcttcaacaaaagttttattcaatactattttaaaaataacagtgttactcgttcatttgatttgttaataagactgatttacccactttttagttcctataatcatacagatttctcgtcctaaatctatttttattgttatttttactttagttttcacttttgctttcagttttttctttttttagcacaagagaaaagtattcagctcataaataattaatatatccatatatcatgcaaactataaaatgtgttgttgtcggtgatggtgccgttggtaaaacttgcttattaatctcgtataccactagtaaatttccagctgattatgttcctactgtttttgataattatgctgtaaccgtgatgataggagacgaaccatttaccttgggattatttgatactgctggtcaagaagattacgacagattaaggcctttgtcatatccatcgactgatgtattccttgtttgtttttccgtcatttctcccgcttcgtttgaaaatgttaaagaaaaatggttcccagaagttcatcaccattgtcccggtgtgccaataattattgtcggtacccaaactgatttacgaaacgatgatgttattttacagagattgcacagacaaaaattgtccccaatcacccaggaacagggtgaaaaattggctaaggaattgagagctgtcaagtatgttgagtgttctgcattgactcaaagaggattgaaaacagtgtttgacgaggctatagtagctgcattagaacctcctgtaattaaaaaatcgaaaaagtgtactattttataggtcggcgatactagaagatagaggatattggaaatagggcatacatgagatattgaatatctatcattaaatatataattagtttttttctaaaacctatctttaggtttgatctcgtttgatgtgttgggctgtttcgcaaaacagtgttccaatcaataaaaagatgtgtgtaagactctaga

SEQ ID NO: 10 - protein, 191 aa, Candida albicans
MQTIKCVVVGDGAVGKTCLLISYTTSKFPADYVPTVFDNYAVTVMIGDEPFTLGLFDTAGQEDYDRLRPLSYPSTDVFLVCFSVISPASFENVKEKWFPEVHHHCPGVPIIIVGTQTDLRNDDVILQRLHRQKLSPITQEQGEKLAKELRAVKYVECSALTQRGLKTVFDEAIVAALEPPVIKKSKKCTIL

SEQ ID NO: 11 - DNA, 2231 bp, Candida albicans
  CDS (291)...(2195)
aagcttgtttcttatctccttagtatattgttttacaacaccacatacacatacacatatagccttcattagccttcattttgacatatttcaataacaatcaagaactacaagtcataactgacacacatataatatcttaattgttattataaatttattcttgattagattttagacgggcagaaacaaaaacggaaaatccaactcatccccgataactacacacatctatattaaatcatctattagtctatcagttatatctccctccccttttcttctaacaaatgattaagacgtttcggaaaagtaaaagactgtcgagtaattcaagttcacccaagaaaacaatatctcgagtatcatcaacttcaagtaatcaaacatctcatgatggaatattacaatcacctaaaaaagtcattagagctctatatgattatgaacctcaaggtcctggagaattgaaatttttcaaaggagattttttccatgtattaaatgatgttgatgatgaattacataaagaagcggaacgtaatggatggatagaagcaacaaatccaatgactcaacttaaagggatggtccccattagttattttgaaatatttgatcgatctcgtcctacagttacagcatcatcaaacagttttacaaattccattgatattcaacatcaacatcaacaaggaattcacaatggaacaggaaatcgaaatttaaatcaaacattatatgctgttacactatatgaatttaaagctgaacgagatgatgaattggatataatgcctaatgaaaatttaattatttgtgcacatcatgattatgaatggtttattgccaaaccaataaatcgattaggtggaccaggtttagtacctgtttcttatgttaaaataattgatcttttaaaccctaattctcattatacatcaattgatacatcaaggcgatcacaagtcatacaagtaatcaatggatttaatataccgacagtagaacaatggaaaaatcaaactgccaaatatcaagcttcaacaatcccccttggttcaatatcaggaagtggtactccaccaacatcagctaattcacaatattttgataatcatactatgacttcaaatcgatcatcactgggttcatcaatttctattattgaagctagtgttgattcatatcaattagatcatggtcgatatcaatattcaataactgctcgattaaataatggcagaataagatatttatatcgatattatcaagatttttatgatttacaagtgaaattattagaattatttccttatgaagctgggagaattgaaaattctaaaagaataattccatctataccaggacctttaattaatgtcaatgattcaatatcaaaattacgaagagaaaaattggattattatttatcaaatttaattgcattacctagtcatatatctcgatcagaagaagtattaaaattatttgatgttttagataatggatttgatcgagaaactgatgctattaataaacgattttctaaaccaataagtcaaaaatcaaattctcatcaagatagattatctcaatattccaattttaacgttttacaacaacaacaacaacaacagcaacaacagcaatatgctcatcattcaagaggttctgataattcacctactaatgaatcatcaggttcaaatttaattaattcttcttctcataatgattcatcattatcttcatcaccaccaccaccaccaccacaaactgtcaccaccacgaacaccacgaacaccaccataaccacagactcctcatcaaaacaaccaaaagccaaagtgaaattttattttgatgatgatatatttgtattattaatcccaaccaatttacgattacaagatttaaaatcaaaattatttaaacgattagaattggatattacttataaatatgaaaaacctgatcaacaacaaaaacctacatcagaatcaattcatttatttttgaaaaatgattttgaagattttttaattgaaaatgaaactagcaacaacaacaatctggaaattgatttcgaaaatgaaattattaaagaaaaattaggagaatttgaagttaatgatgatgaaaaatttcaaagtattttatttgataaatgtaaattaatggttttagtatattaaacagagatcaataagagagagagagagagacat

SEQ ID NO: 12 - protein, 635 aa, Candida albicans
MIKTFRKSKRLSSNSSSPKKTISRVSSTSSNQTSHDGILQSPKKVIRALYDYEPQGPGELKFFKGDFFHVLNDVDDELHKEAERNGWIEATNPMTQLKGMVPISYFEIFDRSRPTVTASSNSFTNSIDIQHQHQQGIHNGTGNRNLNQTLYAVTLYEFKAERDDELDIMPNENLIICAHHDYEWFIAKPINRLGGPGLVPVSYVKIIDLLNPNSHYTSIDTSRRSQVIQVINGFNIPTVEQWKNQTAKYQASTIPLGSISGSGTPPTSANSQYFDNHTMTSNRSSLGSSISIIEASVDSYQLDHGRYQYSITARLNNGRIRYLYRYYQDFYDLQVKLLELFPYEAGRIENSKRIIPSIPGPLINVNDSISKLRREKLDYYLSNLIALPSHISRSEEVLKLFDVLDNGFDRETDAINKRFSKPISQKSNSHQDRLSQYSNFNVLQQQQQQQQQQQYAHHSRGSDNSPTNESSGSNLINSSSHNDSSLSSSPPPPPPQTVTTTNTTNTTITTDSSSKQPKAKVKFYFDDDIFVLLIPTNLRLQDLKSKLFKRLELDITYKYEKPDQQQKPTSESIHLFLKNDFEDFLIENETSNNNNLEIDFENEIIKEKLGEFEVNDDEKFQSILFDKCKLMVLVY

(19) United States
(12) Patent Application Publication
(10) Pub. No.: US 2003/0166886 A1
(43) Pub. Date: Sep. 4, 2003

(54) Candida albicans proteins associated with virulence and hyphal formation and uses thereof

(76) Inventors: Ekkehard Leberer, Beaconsfield (CA); David Y. Thomas, Montreal West, CA (US)

Correspondence Address: OGILVY RENAULT, 1981 MCGILL COLLEGE AVENUE, SUITE 1600, MONTREAL, QC, H3A2Y3, CA

(21) Appl. No.: 10/093,524

(22) Filed: Mar. 11, 2002

Related U.S. Application Data
(63) Continuation-in-part of application No. 09/301,132, filed on Apr. 28, 1999, now abandoned.
Application No. 09/301,132 is a continuation of application No. PCT/CA97/00809, filed on Oct. 29, 1997.
(60) Provisional application No. 60/029,458, filed on Oct. 30, 1996.

(51) Int. Cl. C07H 21/02; C07H 21/04

(52) U.S. Cl. 536/23.1

(57) ABSTRACT

The present invention relates to Candida albicans proteins, such as CaCla4p, Cst20p, CaCdc42p and CaBem1p, associated with virulence and hyphal formation and uses thereof, such as to design screening tests for inhibitors for the treatment of pathogenic fungi infections and/or inflammation conditions. The invention also relates to an in vitro screening test for compounds to inhibit the biological activity of at least one protein selected from the group consisting of CaCla4p, Cst20p, CaCdc42p and CaBem1p, which comprises: a) at least one of said proteins; and b) means to monitor the biological activity of said at least one protein; thereby compounds are tested for their inhibiting potential.

This application is a continuation-in-part of U.S. Ser. No. 09/301,132 filed Apr. 28, 1999, which is a continuation of PCT/CA97/00809 filed Oct. 29, 1997 designating the United States and claiming priority from U.S. provisional patent application Ser. No. 60/029,458 filed Oct. 30, 1996.

### BACKGROUND OF THE INVENTION

1. Field of the Invention

The invention relates to Candida albicans proteins, such as CaCla4p, Cst20p, CaCdc42p and CaBem1p, associated with virulence and hyphal formation and uses thereof, such as to design screening tests for inhibitors for the treatment of pathogenic fungi infections and/or inflammation conditions.

2. (b) Description of Prior Art

Candida albicans is the major fungal pathogen in humans, causing various forms of candidiasis. The incidence of infections is increasing in immunocompromised patients. This fungus is diploid and is capable of a morphological transition from a unicellular budding yeast to a filamentous form. Extensive filamentous growth leads to the formation of a mycelium displaying hyphae with branches and lateral buds. In view of the observation that hyphae seem to adhere to and invade host tissues more readily than does the yeast form, the switch from the yeast to the filamentous form probably contributes to the virulence of this organism (for a review see Fidel, P. L. & Sobel, J. D. (1994) Trends Microbiol. 2, 202-205). The molecular mechanisms by which morphological switching is regulated are poorly understood (Whiteway review 2000, Curr. Op. Microbio., 3:582-588).

Like C. albicans, bakers yeast Saccharomyces cerevisiae is also a dimorphic organism capable of switching under certain nutritional conditions from a budding yeast to a filamentous form. Under the control of nutritional signals, diploid cells switch to pseudohyphal growth (Gimeno, C. J. et al. (1992) Cell 68, 1077-1090), and haploid cells to invasive growth (Roberts, R. L. & Fink, G. R. (1994) Genes Dev. 8, 2974-2985).

The similarities between the dimorphic switching of S. cerevisiae and C. albicans suggest that these morphological pathways may be regulated by similar mechanisms in both organisms. In S. cerevisiae, morphological transitions are controlled by signaling components that are also involved in the mating response of haploid cells (Roberts, R. L. & Fink, G. R. (1994) Genes Dev. 8, 2974-2985; Liu, H. et al. (1993) Science 262, 1741-1744). The switch to pseudohyphal growth requires a transcription factor encoded by the STE12 gene, and a mitogen-activated protein (MAP) kinase cascade including Ste7p (a homolog of MAP kinase kinase or MEK), Ste11p (a MEK kinase homolog) and Ste20p (a MEK kinase kinase) (Roberts, R. L. & Fink, G. R. (1994) Genes Dev. 8, 2974-2985; Liu, H. et al. (1993) Science 262, 1741-1744). The MAP kinases involved in this response are as yet unknown (Roberts, R. L. & Fink, G. R. (1994) Genes Dev. 8, 2974-2985; Liu, H. et al. (1993) Science 262, 1741-1744).

Members of the Ste20p family of serine/threonine protein kinases are thought to be involved in triggering morphogenetic processes in response to external signals in organisms ranging from yeast to mammalian cells. Two of these kinases, Ste20p and Cla4p, are well characterized in S. cerevisiae (Leberer, E. et al. (1992) EMBO J. 11, 4815-4824; Cvrckova, F. et al. (1995) Genes Dev. 9, 1817-1830). Ste20p is required for pheromone signal transduction (Leberer, E. et al. (1992) EMBO J. 11, 4815-4824) and for filamentous growth in response to nitrogen starvation (Roberts, R. L. & Fink, G. R. (1994) Genes Dev. 8, 2974-2985; Liu, H. et al. (1993) Science 262, 1741-1744), and shares an essential function with Cla4p during budding (Cvrckova, F. et al. (1995) Genes Dev. 9, 1817-1830). Ste20p and Cla4p interact with the small G-protein Cdc42p, and this interaction is required for viability of S. cerevisiae cells. Ste20p also interacts with the SH3 domain protein Bem1p, and this interaction plays a role in morphogenetic processes (Leeuw, T. et al. (1995) Science 270, 1210-1213).

Given the increase in the incidence of candidiasis, particularly in immunocompromised patients, it would be desirable to develop a means to screen and identify potential inhibitors of C. albicans.

### SUMMARY OF THE INVENTION

It has now been found that Cst20p, a C. albicans homolog of the Ste20p protein kinase, is required for hyphal growth of C. albicans under certain in vitro conditions. Cst20p has also been shown to play a role in virulence, as judged from significantly prolonged survival of mice infected with CST20 deleted cells. Cst20p, thus, appears to act in a regulatory pathway that is involved in hyphal growth of C. albicans.

It has also been found that CaCla4p, a C. albicans homolog of the Cla4p protein kinase, is required for hyphal formation in vitro in response to serum, and in vivo in a mouse model for systemic candidiasis. CaCla4p is required for efficient colonization of kidneys with C. albicans cells after infection of mice and essential for virulence in the mouse model.

Accordingly, in one aspect of the present invention, there is provided isolated Candida albicans proteins, such as CaCla4p, Cst20p, CaCdc42p and CaBem1p, identified by amino acid sequences as well as by the nucleotide sequences encoding them.

In another aspect of the present invention, there is provided an in vitro screening test useful to identify potential anti-fungal compounds. The assay comprises the steps of:

a) combining at least one protein selected from the group consisting of CaCla4p (SEQ ID NO:8), Cst20p (SEQ ID NO:6), CaCdc42p (SEQ ID NO:10) and CaBem1p (SEQ ID NO:12) with a target that interacts with said protein in a protein/target interaction;

b) adding a test compound to the protein/target mixture; and

c) measuring the protein/target interaction, wherein a lack of protein/target interaction indicates that said compound is a potential anti-fungal compound.

In another aspect, the in vitro screening assay may further comprise the step of:

d) comparing the protein/target interaction in the presence or absence of a test compound, wherein a reduced protein/target interaction in the presence of a test compound indicates that said test compound is a potential anti-fungal compound.

In accordance with another embodiment of the present invention, an assay is provided by which inhibitors of the interactions between CaCla4p and CaCdc42p can be identified, as well as inhibitors of the interactions between Cst20p and CaCdc42p.

The term “fungi” when used herein is intended to mean any fungi, pathogenic or not, which show hyphal induction using kinases, such as C. albicans, Saccharomyces cerevisiae, Aspergillus, Ustilago maydis, and all the species of the fungal genera Aspergillus, Blastomyces, Candida, Cladosporium, Coccidioides, Cryptococcus, Epidermophyton, Exophilia, Fonsecaea, Histoplasma, Madurella, Malassezia, Microsporum, Paracoccidioides, Penicillium, Phaeoannellomyces, Phialophora, Scedosporium, Sporothrix, Torulopsis, Trichophyton, Trichosporon, Ustilago, Wangiella, Xylohypha, among others.

### BRIEF DESCRIPTION OF THE DRAWINGS

FIGS. 1A to 1D illustrate photomicrographs which show that C. albicans CST20 gene complements defects in pseudohyphal growth of ste20/ste20 S. cerevisiae diploid cells.

FIGS. 2A to 2C show the morphology of S. cerevisiae MATα cells (strain YEL306-1A) deleted for STE20 and CLA4, and transformed with plasmids expressing CLA4 (FIG. 2A), STE20 (FIG. 2B) and C. albicans CST20 (FIG. 2C).

FIG. 3 shows the nucleotide (SEQ ID NO:5) and predicted amino acid sequences of CST20 (SEQ ID NO:6).

FIG. 4A is the deletion of CST20 in C. albicans.

FIG. 4B is the Southern blot analysis with a CST20 fragment from EcoRI to XbaI as a probe.

FIGS. 5A to 5D show colonies of C. albicans cells grown for 5 days at 37° C. on solid “spider” medium containing mannitol. Wild type strain SC5314 (A), ura3/ura3 cst20Δ/cst20Δ::URA3 strain CDH22 (B), ura3/ura3 cst20Δ/cst20Δ::CST20::URA3 strain CDH36 (obtained by reintegration of CST20 into strain CDH25 by homologous recombination using linearized plasmid pDH190) (C), ura3/ura3 cst20Δ/cst20Δ strain CDH25 transformed with plasmids pYPB1-ADHpt (D). Photomicrographs of representative colonies were taken with a 2× lens (bar=2 mm).

FIGS. 6A to 6C illustrate virulence assays. Survival curves of mice (n=10 for each C. albicans strain at each inoculation dose) infected with 1×10⁶ (A) and 1×10⁵ (B) cells of C. albicans strains SC5314 (wild type), CAI4 (ura3/ura3), CDH22 (ura3/ura3 cst20Δ/cst20Δ::URA3) (C) Staining of mouse kidney sections with periodic acid Schiff's stain 48 hours after infection with cst20Δ/cst20Δ::URA3 mutant strain CDH22 (a). Some hyphal cells are indicated with arrows (bar=0.1 mm).

FIG. 7 illustrates the nucleotide (SEQ ID NO:7) and predicted amino acid (SEQ ID NO:8) sequences of CaCLA4.

FIG. 8A illustrates the deletion of CaCLA4 in C. albicans.

FIG. 8B illustrates the Southern blot analysis with the CaCLA4 fragment from PstI to XbaI as a probe.

FIG. 8C illustrates the Northern blot analysis with the CaCLA4 fragment as a probe. PCR with the divergent oligodeoxynucleotides OEL109 and OEL110 was used to delete the coding sequence of CaCLA4. A hisG-URA3-hisG cassette was then inserted, and homologous recombination was used in a two-step procedure to replace both CaCLA4 alleles.

FIG. 9 illustrates virulence assays. Survival curves of mice (n=15 for each C. albicans strain) infected with 1×10⁶ cells of C. albicans strains SC5314 (wild-type), CDH77 (CaCLA4/cacla4Δ), CLJ1 (cacla4Δ/cacla4Δ) and CLJ5 (CaCla4Δ/cacla4Δ) transformed with the control plasmid pVEC and plasmid pVEC-CaCLA4 carrying the CaCLA4 gene.

FIG. 10 illustrates the staining of mouse kidney sections with periodic acid Schiff's stain 48 h after infection with C. albicans strains SC5314 and CLJ1.

FIG. 11 illustrates the nucleotide (SEQ ID NO:9) and predicted amino acid (SEQ ID NO:10) sequences of CaCdc42p.

FIG. 12 illustrates the nucleotide (SEQ ID NO:11) and predicted amino acid (SEQ ID NO:12) sequences of CaBem1p.

### DETAILED DESCRIPTION OF THE INVENTION

The isolation of proteins that play a role in the viability and/virulence of Candida albicans allows for the development of an assay useful to screen for potential inhibitors of Candida albicans. Each one of the C. albicans' proteins, CaCla4p, Cst20p, Cdc42p and Bem1p, has been found to play such a role. Accordingly, the determination of compounds capable of inhibiting one or more of these proteins would be beneficial in the development of potential anti-fungal agents.

The CST20 gene of Candida albicans was cloned by functional complementation of a deletion of the STE20 gene in Saccharomyces cerevisiae. CST20 encodes a homolog of the Ste20p/p65^{PAK} family of protein kinases. Colonies of C. albicans cells deleted for CST20 revealed defects in the lateral formation of mycelia on synthetic solid “Spider” media. However, hyphal development was not impaired in some other media. Cells deleted for CST20 were less virulent in a mouse model for systemic candidiasis. Our results suggest that more than one signaling pathway can trigger hyphal development in C. albicans, one of which has a protein kinase cascade that is analogous to the mating response pathway in S. cerevisiae and might have become adapted to the control of mycelial formation in asexual C. albicans.

The CaCLA4 gene of C. albicans was cloned by functional complementation of the growth defect of S. cerevisiae cells deleted for the STE20 and CLA4 genes. CaCLA4 encodes a homolog of the Ste20p family of serine/threonine protein kinases with pleckstrin homology and Cdc42p binding domains in the amino-terminal noncatalytic region. Deletion of both alleles of CaCLA4 in C. albicans caused defects in hyphal formation in vitro in synthetic liquid and solid media, and in vivo in a mouse model for systemic candidiasis. The deletions reduced the invasion of C. albicans cells into kidneys after infection into mice and completely suppressed virulence in the mouse model. Thus, hyphal formation of C. albicans mediated by the CaCla4p protein kinase may contribute to the pathogenicity of this dimorphic fungus.

The CaBEM1 and CaCDC42 genes of C. albicans were cloned by functional complementation of the growth defect of S. cerevisiae cells deleted for the BEM1 and CDC42 genes, respectively. CaBEM1 encodes an SH3 domain protein with homology to Bem1p, and CaCDC42 encodes a small G-protein with homology to members of the Rho-family of G-proteins.

With isolation of genes encoding the present C. albicans proteins, namely CaCla4p, Cst20p, Cdc42p, and Bem1p, purified proteins can be made for use in inhibitor assays in accordance with the present invention. Examples of assays utilizing these proteins to identify compounds that inhibit the inherent protein/target interactions of these proteins are described in detail in the specific examples provided herein. As will be appreciated, the protein/target interactions in which these proteins are involved include interactions with each other as well as interactions with independent target compounds.

Materials and Methods

Yeast Manipulations

The yeast form of C. albicans was cultured at 30° C. in YPD medium. Hyphal growth was induced at 37° C. on solid “Spider” media (Liu, H. et al. (1994) Science 266, 1723-1726) containing 1% (w/v) nutrient broth, 0.2% (w/v) K₂HPO₄, 2% (w/v) agar and 1% (w/v) of the indicated sugars (pH 7.2 after autoclaving). Cells were grown in liquid “Spider” media at 30° C. to stationary phase, and then incubated for 5 days at 37° C. on solid “Spider” media at a density of about 200 cells per 80 mm plates. All media were supplemented with uridine (25 μg/ml) for the growth of Ura⁻ strains. Germ tube formation was induced at 37° C. in either 10% fetal bovine serum (GIBCO/BRL) on liquid “Spider” media containing the indicated sugars at an inoculation density of 10⁷ cells per ml.

Yeast manipulations were performed according to standard procedures.

Isolation of CST20

The CST20 gene was isolated from a genomic C. albicans library constructed in plasmid YEp352 from genomic DNA of the clinical isolate WO1 (Boone, C. et al. (1991) J. Bacteriol. 173, 6859-6864). A plasmid carrying an amino-terminally truncated version of CST20 missing the first 918 nucleotides of coding sequence was isolated by screening for suppressors of defects in basal FUS1::HIS3 expression and mating in S. cerevisiae strain YEL64 which was disrupted in STE20. A fragment from nucleotides 958 to 1,252 of CST20 was amplified by the polymerase chain reaction (PCR) and used as a probe to isolate a full length clone by colony hybridization to the C. albicans genomic library transformed into E. coli strain MC1061. Both DNA strands were sequenced by the dideoxy chain termination method. The full-length clone was subcloned between the SacI and HindIII sites of the S. cerevisiae centromere plasmid pRS316 to yield plasmid pRL53.

Isolation of CaCLA4

A C. albicans homolog of the S. cerevisiae CLA4 gene was cloned by functional complementation of the growth defect of S. cerevisiae cells that were deleted for the STE20 and CLA4 genes.

The S. cerevisiae MATα strain YEL257-1A-2 deleted for STE20 and CLA4 and carrying plasmid pDH129 with CLA4 under control of the GAL1 promoter was transformed with the genomic C. albicans library constructed in the S. cerevisiae vector YEp352 carrying URA3 as selectable marker (Boone, C. et al. (1991) J. Bacteriol. 173, 6859-6864). Transformants were grown on selective medium in 4% galactose and then replica-plated to selective medium containing 2% glucose to select for plasmids that were able to support growth in the absence of Cla4p and Ste20p. By screening 1,600 transformants, we isolated plasmid YEp352-CaCLA4 carrying an insert of 5.6 kb with an open reading frame of 2,913 bp capable of encoding a homolog of Cla4p. Subcloning indicated that this open reading frame was responsible for complementation. Both DNA strands were sequenced by the dideoxy chain termination method.

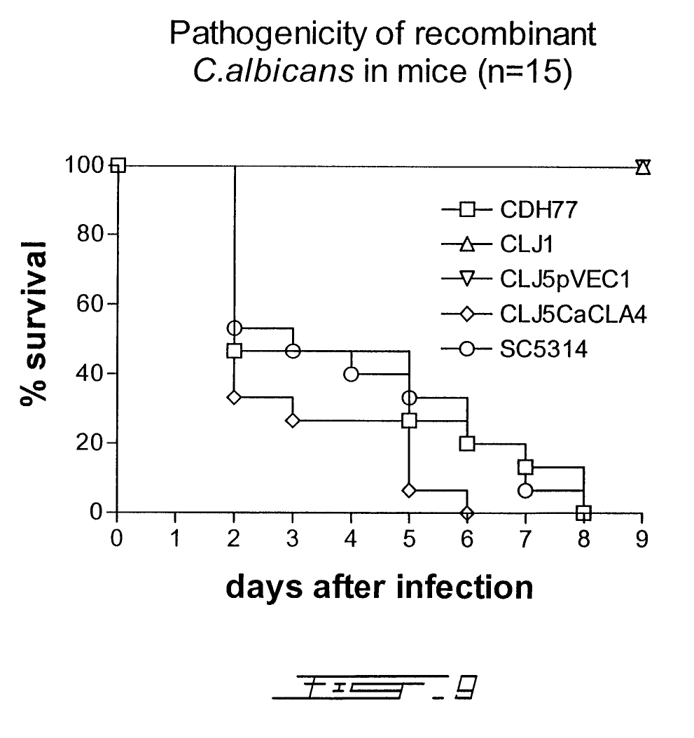

The open reading frame of the CaCLA4 gene is capable of encoding a protein of 971 amino acids with a predicted molecular weight of 107 kDa and a domain structure characteristic of the Ste20p family of protein kinases (FIG. 7). The catalytic domain present in the carboxyl terminal half of the protein has sequence identities of 74, 63 and 64%, respectively, with S. cerevisiae Cla4p, S. cerevisiae Ste20p and an uncharacterized open reading frame present in the S. cerevisiae genome, 65% with the C. albicans Ste20p homolog Cst20p, and 61% with rat p₆₅PAK (FIG. 7). The amino terminal, noncatalytic region contains a sequence from amino acid residues 69 to 180 with similarity to pleckstrin homology (PH) domains and a sequence from amino acid residues 229 to 292 with 63% identity to the Cdc42p binding domain of S. cerevisiae Cla4p that has been shown to bind the small GTPase Cdc42p (Cvrckova, F. et al. (1995) Genes Dev. 9, 1817-1830). The remaining noncatalytic sequences are less conserved.

Isolation of CaCDC42

The S. cerevisiae MATα strain DJTD2-16A carrying the cdc42-1^{ts} mutation was transformed with the genomic C. albicans library constructed in the S. cerevisiae vector YEp352 carrying URA3 as selectable marker (Boone, C. et al. (1991) J. Bacteriol. 173, 6859-6864). Transformants were grown on selective medium at room temperature. Colonies were then replica-plated to selective medium and grown at 34° C. By screening 2,000 transformants, we isolated plasmid YEp352-CaCDC42 carrying an open reading frame of 573 bp capable of encoding a homolog of Cdc42p. Both DNA strands were sequenced by the dideoxy chain termination method. Sub-cloning of various restriction endonuclease fragments indicated that the open reading frame was responsible for complementation of the temperature-sensitive growth defect caused by the cdc42-1^{ts} mutation.

Construction of C. albicans Strains and Plasmids

To construct a CST20 null mutant, an EcoRI to SacI fragment from nucleotide positions 989 to 4,134 of CST20 was subcloned into the Bluescript KS(+) vector (Stratagene) to yield plasmid pDH119. A plasmid that contained CST20-flanking sequences from nucleotides 989 to 1,674, and 3,423 to 4,134 joined with BamHI sites, was then created by PCR using the divergent oligodeoxynucleotide primers ODH68 (5′-CGGGATCCAGACCAACCACTCGAACTACT-3′ (SEQ ID NO:1) and ODH69 (5′-CGGGATCCGAAGGTGAACCACCATATTTG-3′ (SEQ ID NO:2); newly introduced BamHI sites are underlined) and plasmid pDH119 as a template. The amplified DNA was cleaved with BamHI and ligated with a 4 kb BamHI to BglII fragment of a hisG-URA3-hisG cassette derived from plasmid pCUB-6 (Fonzi, W. A. & Irwin, M. Y. (1993) Genetics 134, 717-728) to yield plasmid pDH183. This plasmid was linearized with XhoI and SacI and transformed into the Ura⁻ C. albicans strain CAI4 (Fonzi, W. A. & Irwin, M. Y. (1993) Genetics 134, 717-728) to partially replace the coding region of one of the chromosomal CST20 alleles with the hisG-URA3-hisG cassette by homologous recombination. Ura⁺ transformants were selected on Ura⁻ medium, and integration of the cassette into the CST20 locus was verified by Southern blot analysis. Spontaneous Ura⁻ derivatives of two of the heterozygous disruptants were selected on medium containing 5-fluoroorotic acid. These clones were screened by Southern blot hybridization to identify those that had lost the URA3 gene by intrachromosomal recombination mediated by the hisG repeats. This procedure was then repeated to delete the remaining functional allele of CST20.

A similar procedure was employed to delete the CaCLA4 gene. A 4.6 kb XbaI fragment of YEp352-CaCLA4 was subcloned into the pBluescript KS(+) vector (Stratagene) to yield plasmid pDH205. A plasmid that contained CaCLA4 flanking sequences joined with BglII sites was then created by PCR using the divergent oligodeoxynucleotide primers OEL109 (5′-GAAGATCTTGTAATCAATGTTCCCGTGGA-3′ (SEQ ID NO:3) and OEL110 (5′-GAAGATCTCATCGTGATATTAAATCCGAT-3′ (SEQ ID NO:4); newly introduced BglII sites are underlined) and plasmid pDH205 as template. The amplified DNA was cleaved with BglII and ligated with a 4 kb BamHI-BglII fragment of a hisG-URA3-hisG cassette derived from plasmid pCUB-6 (Fonzi, W. A. & Irwin, M. Y. (1993) Genetics 134, 717-728) to yield plasmid pDH210. This plasmid was linearized with PstI and SacI and transformed into the Ura⁻ C. albicans strain CAI4 (Fonzi, W. A. & Irwin, M. Y. (1993) Genetics 134, 717-728) to replace the coding region of one of the chromosomal CaCLA4 alleles with the hisG-URA3-hisG cassette by homologous recombination. Ura⁺ transformants were selected on Ura⁻ medium, and integration of the cassette into the CaCLA4 locus was verified by Southern blot analysis. Spontaneous Ura⁻ derivatives were then selected on medium containing 5-fluoroorotic acid. These clones were screened by Southern blot hybridization to identify those that had lost the URA3 gene by intrachromosomal recombination mediated by the hisG repeats. This procedure was then repeated to delete the remaining functional allele of CaCLA4.

To reintegrate CST20 into the genome of mutant strains, the C. albicans integration plasmid pDH190 was constructed by subcloning a KpnI to PstI fragment of CST20 into pBS-cURA3 (pBluescript KS(+) into which the C. albicans URA3 gene was cloned between the NotI and XbaI sites of the polylinker). The integration plasmid was then linearized with NsiI and transformed into C. albicans to target integration into the NsiI site of the CST20Δ::hisG fusion gene. Integrations were selected on Ura⁻ medium and confirmed by Southern blot analysis.

The C. albicans CST20 expression plasmid pDH188 was constructed by subcloning a SacI to PstI fragment of CST20 into plasmid pVEC carrying a C. albicans autonomously replicating sequence and URA3 as selectable marker. The C. albicans plasmid pVEC-CaCLA4 was constructed by subcloning the KpnI to SacI insert of YEp 352-CaCLA4 into plasmid pVEC.

Methods Associated with CaCDC42

Site-directed mutagenesis using the Quickchange Site-Directed Mutagenesis kit from Stratagene was used to generate the following single and double point mutants in CaCDC42 in pJA19; G12V (pSU7, using oligonucleotides OSU5 and OSU6), D118A (pSU9, using oligonucleotides OSU7 and OSU8), C188S (pSU13, using oligonucleotides OSU9 and OSU64), G12V C188S (pSU16) and D118A C188S (pSU18). All constructs were verified by sequence analysis. To overexpress and integrate wild type CaCDC42 and the five mutant CaCDC42 versions in C. albicans, a cassette was made containing 3 kb of hisGURA3hisG sequences (providing an excisable C. albicans selectable marker) isolated from pCUB− (Fonzi and Irwin, 1993) and the 1.4 kb PCK1 promoter (a C. albicans regulatable promoter) isolated from pCA01 (Leuker et al., 1997) yielding pJA24. PCK1 encodes PEP carboxykinase, an enzyme strongly repressed in glucose-containing media (Leuker et al., 1997). PCK1 expression can be induced in media containing a gluconeogenic carbon source such as 2% casamino acids (CAA) media (Leuker et al., 1997). This cassette was cloned into the unique BamHI site in pJA19 and pSU7, 9, 13, 16, 18 generating pJA28 and pSU48, 50, 51, 52, and 45, respectively. Each plasmid was linearized using a unique HpaI site within the PCK1 promoter and was transformed into strains CAI4 (ura3/ura3—Fonzi and Irwin, 1993) or CaDH85 (ura3/ura3CaCDC42/cacdc42::hisG) by either the LiOAc procedure (Schiestl et al., 1993) or the 1-step transformation protocol (Chen et al., 1992). In addition, pJA28 and pSU48 were integrated into a doubly deleted CST20 and CaCLA4 strains CaDH25 and CaLJ5 respectively to create CaSU112 (CST20 double deletion with PCK1CDC42) and CaSU116 (CST20 double deletion with PCK1CDC42^{G12V}) as well as CaSU138 (CaCLA4 double deletion with PCK1CDC42) and CaSU142 (CaCLA4 double deletion with PCK1CDC42^{G12V}). Correct integration was verified by isolating genomic DNA and using PCR with primers OSU43 and OSU45. Integration of PCK1CaCDC42 derivatives was further verified by Southern analysis using the DIG kit from Boehringer Mannheim.

Construction and Analysis of a CaCDC42 Double Deletion in a PCK1-CDC42 Containing Strain

A deletion cassette was constructed by removing 640 bp between the KpnI and XbaI sites in pDH208 and replacing this with a BamHI site using oligonucleotides OEL112 plus OEL113, and inserting the hisGURA3hisG cassette to yield pDH212. C.albicans strains CaDH85 and CaSU92 (PCK1CaCDC42) were transformed by either LiOAc procedure or by 1-step protocol using the 9.5 kb SacI-HindIII deletion fragment from pDH212 that contains 478 bp CaCDC42 upstream sequences and coding region, the URA blaster cassette, and 1.8 kb of downstream sequences. Deletion of the second CaCDC42 allele was screened by PCR (three independent products from OSU44 plus OSU8, OSU33 plus OSU34, and ODH103 plus OSU44) and confirmed by Southern analysis; three independent knockouts were found in the first 30 transformants screened in CaSU92 (PCK1CaCDC42) yielding CaSU96-98. No double knockouts were obtained in the 150 CaDH85 transformants screened.

Phenotypic Analysis

To assess the effect of Cdc42p depletion on vegetatively grown C. albicans cells, all three of strains CaSU96-98 were grown overnight in 2% CAA (casamino acids) YNB (yeast nitrogen base, Difco)-ura media at 30° C. After 18 hrs of growth the cells were subcultured in either 2% glucose-YNB-ura or 2% CAA YNB-ura and aliquots were stained with DAPI and examined microscopically at various timepoints for up to 10 hrs. To assess the effect of CaCdc42p depletion on hyphal induction, cells were grown for 18 hrs in 2% CAA-ura and were then subcultured under the following conditions: 2% CAA-ura+10% serum at 37° C.; 2% glucose+10% serum at 37° C., and 2% glucose for 4 hrs at 30° C., followed by the addition of 10% serum and incubation at 37° C. Aliquots of each culture were stained with DAPI and examined microscopically every 2-3 hrs.

FACS Analysis

Cells were grown in the specified medium and fixed with 70% ethanol for 20 minutes. They were then resuspended in 1 ml of PBS containing 10 μg/ml propidium iodide (Molecular Probes, Eugene, Oreg., USA). The analysis was performed on an EPICS® XL-MCL flow cytometer (Beckman-Coulter) using a 488 nm dichroic filter for side scattering detection and a 620 nm band pass for propidium iodide fluorescence detection. Forward scattering (FS) and side scattering (SS) data were expressed as mean relative units and propidium iodide as mean fluorescence unit, both on a linear scale. Approximately 30,000 cells were analyzed per experiment to monitor the size of CaSU96 and CaSU97 cells either 6 hours or 18 hours after subculture into 2% glucose-ura or 2% CAA-ura.

PCK1 CaCDC42 Point Mutant Overexpression

A series of point mutants in CaCDC42 were made in order to assess their function in C. albicans morphogenesis. The point mutations chosen are well defined and the effects have been investigated in other systems (Johnson, 1999). These included the G12V, the D118A and the C188S changes, as well as the G12V-C188S and D118A-C188S double mutants. To assess the effect of the overexpression of PCK1CaCDC42 wild type and mutants, each strain was grown overnight in liquid YNB 2% glucose-ura or YNB 2% CAA-ura. For solid plating the cells were grown in liquid YNB 2% glucose-ura overnight then washed and diluted 10⁻⁵ and 20 μl aliquots were plated onto YNB 2% glucose-ura or YNB 2% CAA-ura and incubated at 30° C. or patched directly on YNB 2% gluc-ura or YNB 2% CAA-ura and incubated at 23° C. For liquid hyphal induction, cells were grown overnight at 30° C. in either YNB 2% glucose-ura or YNB 2% CAA-ura, then subcultured in the same media with the addition of 10% FBS and transferred to 37° C. Hyphal inductions were monitored for up to 6 hrs.

Cell Staining (DAPI and Calcofluor) and Microscopy Nomarski/Fluorescence)

Cells were first fixed in 70% ethanol for 20 minutes and rinsed two times in 10 mM phosphate buffer pH 7.4, 150 mM NaCl (PBS) and resuspended in 500 μl PBS before addition of a 1:1000 dilution of either 1 mg/ml 4′,6′-diamino-2-phenylindole (DAPI) or 1 mg/ml Calcofluor white. Cells were stained with either DAPI for 12 minutes or Calcofluor for 5 minutes, then rinsed two times in PBS and examined on a Zeiss Axiophot microscope with a 40× objective under Nomarski optics or under fluorescence conditions, and photographed.

Molecular Cloning of CaBEM1

A C. albicans homolog of the CaBEM1 gene was cloned by functional complementation of the growth defect of S. cerevisiae cells deleted for the BEM1 gene. This defect was fully complemented by plasmid YEp352-CaBEM1 carrying the CaBEM1 gene. The open reading frame of the CaBEM1 gene is capable of encoding a protein of 635 amino acids with a domain structure characteristic of Bem1p (FIG. 12). CaBem1p contains two conserved SH3 domains, which are most homologous to the SH3 domains of Bem1p, and also has homology to Bem1p outside of the SH3 domains.

The S. cerevisiae MATα strain YEL220-1A deleted for BEM1 and carrying plasmid pGAL-BEM1 with BEM1 under control of the GAL1 promoter was transformed with the genomic C. albicans library constructed in the S. cerevisiae vector YEp352 carrying URA3 as selectable marker (Boone, C. et al. (1991) J. Bacteriol. 173, 6859-6864). Transformants were grown on selective medium in 4% galactose and then replica-plated to selective medium containing 2% glucose to select for plasmids that were capable of supporting growth of Bem1p-depleted cells. We isolated plasmid YEp352-CaBEM1 carrying an open reading frame of 1,905 bp fulfilling this criterion and capable of encoding a homolog of Bem1p. Both DNA strands were sequenced by the dideoxy chain termination method, and subcloning of various restriction endonuclease fragments indicated that this open reading frame was responsible for complementation.

Northern Blot Analyses

Northern blots of total and poly (A)⁺ RNA from C. albicans cells were performed as described (Leberer, E. et al. (1992) EMBO J. 11, 4815-4824). Signals were quantified by 2-D radioimaging.

Animal Experiments

Eight week-old, male CFW-1 mice (Halan-Winkelmann, Paderborn, Germany) were inoculated with 1×10⁵ or 1×10⁶ cells by intravenous injection. Survival curves were calculated according to the Kaplan-Meier method using the PRISM™ program (GraphPad Software Inc., San Diego) and compared using the log-rank test. A P value <0.05 was considered significant.

To quantify colony-forming C. albicans units in kidneys, mice were sacrificed by cervical dislocation 48 hours after injection and kidneys were homogenized in 5 ml phosphate buffered saline, serially diluted and plated on YNG medium (0.67% yeast nitrogen base, 1% glucose, pH 7.0). Histological examination of kidney sections was done with periodic acid Schiff's stain.

Results

Isolation and Characterization of CST20

A C. albicans homolog of the S. cerevisiae STE20 gene was cloned by functional complementation of the pheromone signaling defect of S. cerevisiae cells that were deleted for the STE20 gene. The mating defect of the STE20 deleted S. cerevisiae strain YEL20 was fully complemented by introduction of the centromeric plasmid pRL53 carrying full length CST20 (mating efficiency was 81±9% in cells expressing CST20, compared with 85±8% in cells expressing STE20; n=3). Similarly, defects in growth arrest and morphological changes in response to pheromone were completely cured by transformation with the CST20 plasmid.

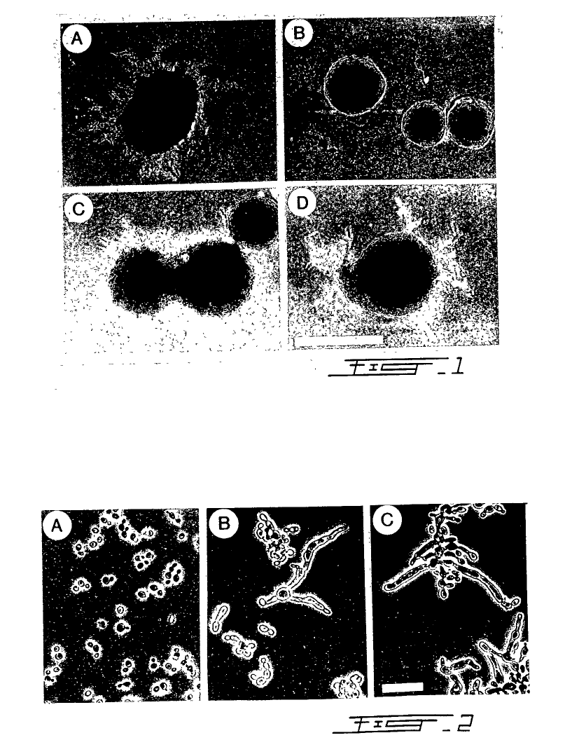

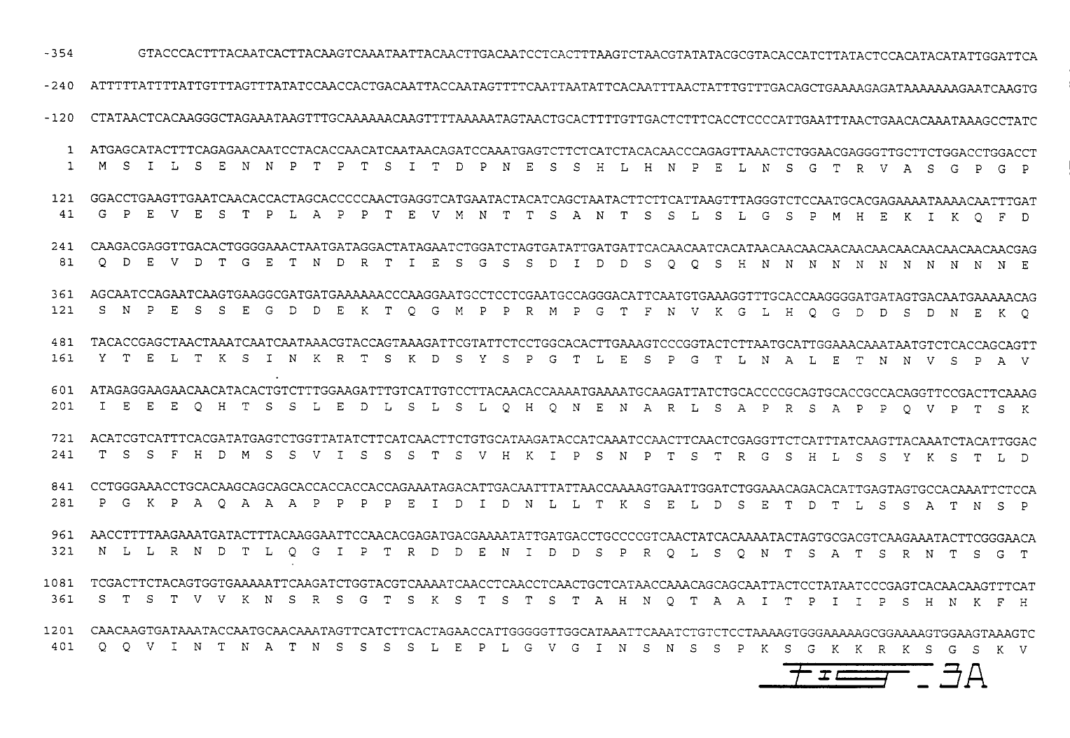

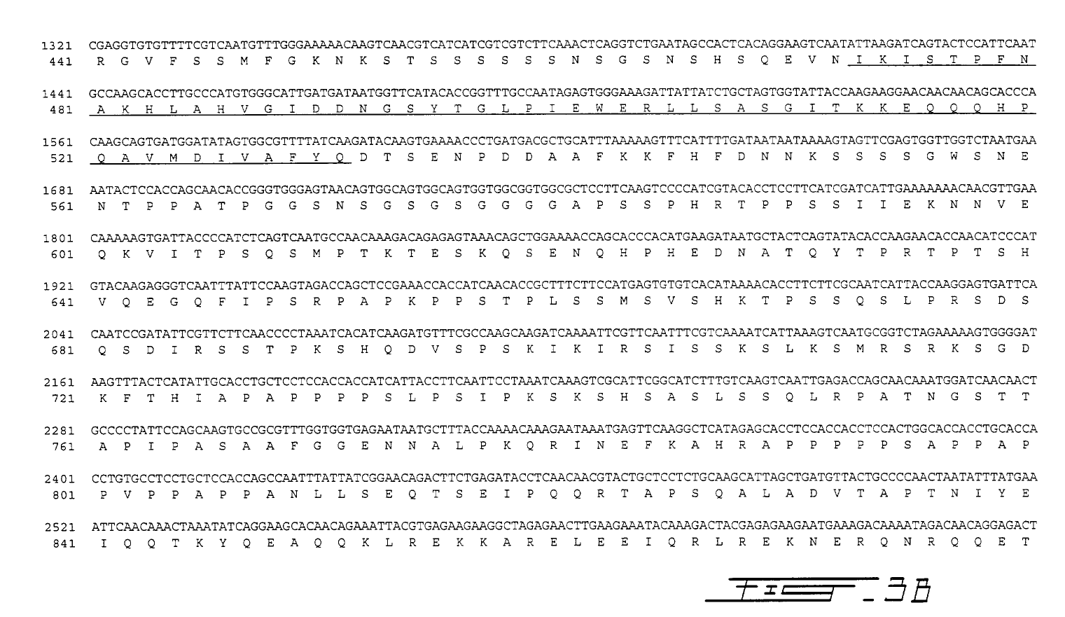

As shown in FIG. 1, nitrogen deficiency-induced pseudohyphae formation, which is blocked by disruption of STE20 in diploid cells (Liu, H., Styles, C. & Fink, G. R. (1993) Science 262, 1741-1744), was restored by introduction of the CST20 plasmid. Colonies of the diploid STE20 wild type strain L5266 (4) (FIG. 1A) and the isogenic ste20/ste20 strain HLY492 (4) transformed with either the control plasmid pRS316 (FIG. 1B), the CST20 plasmid pRL53 (FIG. 1C), or the STE20 plasmid pSTE20-5 (9) (FIG. 1D) were grown on nitrogen starvation medium (2) for 5 days at 30° C. Photomicrographs were taken with a 4× objective (bar=1 mm).

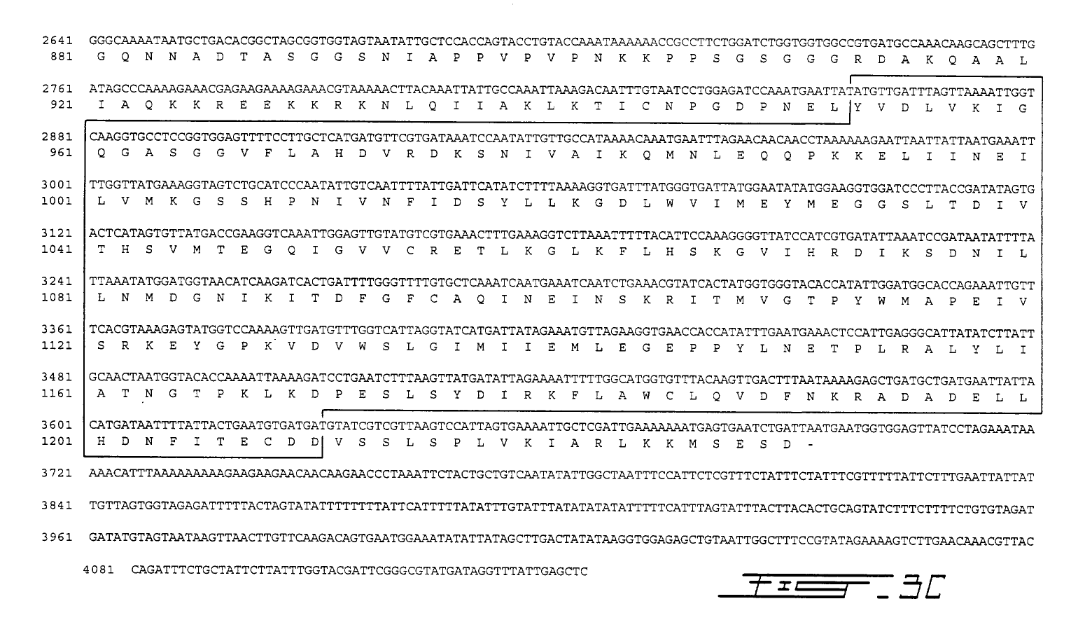

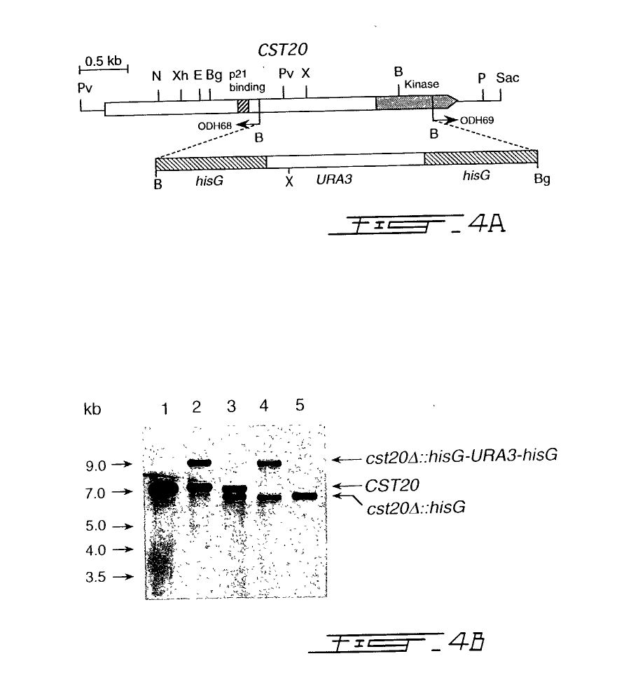

As illustrated in FIG. 2, the cytokinesis defect caused by deletion of CLA4, encoding an S. cerevisiae isoform of Ste20p (Cvrckova, F. et al. (1995) Genes Dev. 9, 1817-1830), was not complemented by CST20 (FIG. 2). However, the lethality caused by deletion of both STE20 and CLA4 (Cvrckova, F. et al. (1995) Genes Dev. 9, 1817-1830), could be rescued by CST20 (FIG. 2). The diploid strain YEL306 heterozygous for ste20Δ::TRP1/STE20 cla4Δ::LEU2/CLA4 was transformed with plasmid pRS316 carrying either no insert, CLA4 (pRL21), CST20 (pRL53) or STE20 (pSTE20-5), and then sporulated and dissected. No viable haploid ste20Δ cla4Δ spores were obtained from transformants with the plasmid without insert, but were obtained from transformants with plasmids carrying CLA4 (FIG. 2A), STE20 (FIG. 2B) or CST20 (FIG. 2C).

Cells were grown to mid-exponential phase in YPD medium at 30° C. No viable ste20Δ cla4Δ segregants were obtained in medium containing 5-fluoro-orotic acid suggesting that the plasmids were essential for viability. Neither STE20 nor CST20 were able to suppress the morphological defect of cla4Δ cells. Photomicrographs were taken by phase contrast with a 40× objective (bar=30 μm).

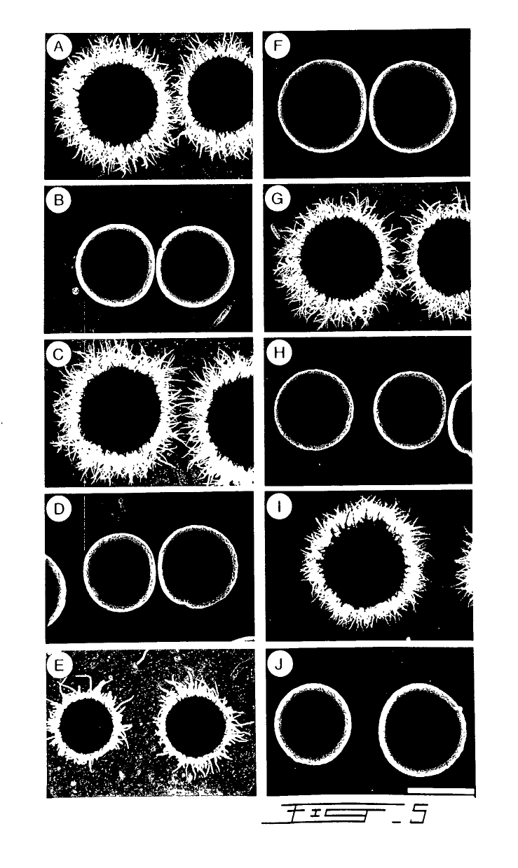

The open reading frame of CST20 is capable of encoding a protein of 1,229 amino acids with a predicted molecular weight of 133 kDa and a domain structure characteristic of the Ste20p/p65^{PAK} family of protein kinases (FIG. 3). Numerals at the left margin indicate nucleotide and amino acid positions (FIG. 3). Nucleotide 1 corresponds to the first nucleotide of the initiation codon and amino acid 1 to the first residue of the deduced protein. The putative p21 binding domain has been shadowed, and the kinase domain has been boxed.

The catalytic domain present in the carboxyl terminal half of the protein has sequence identities of 76 and 56%, respectively, with S. cerevisiae Ste20p (Leberer, E. et al. (1992) EMBO J. 11, 4815-4824) and Cla4p (Cvrckova, F. et al. (1995) Genes Dev. 9, 1817-1830). The amino terminal, non-catalytic region contains a sequence from amino acid residues 473 to 531 with 68% identity to the p21 binding domain of Ste20p that has been shown to bind the small GTPase Cdc42p. This region contains the sequence motif ISxPxxxxHxxH thought to be important for the interaction of the p21 binding domain with the GTP-bound forms of Cdc42Hs and Rac1 (Cvrckova, F. et al. (1995) Genes Dev. 9, 1817-1830). The remaining non-catalytic sequences are less conserved. Unique sequences not present in Ste20p and the other members of the family are found at the amino terminus and between the p21 binding and catalytic domains.

A CST20 transcript of 4.9 kb in size was detected in Northern blots. This transcript was present at similar levels in yeast cells grown in YPD at room temperature and germ tubes induced by a temperature shift to 37° C. Chromosomal Deletion of CST20

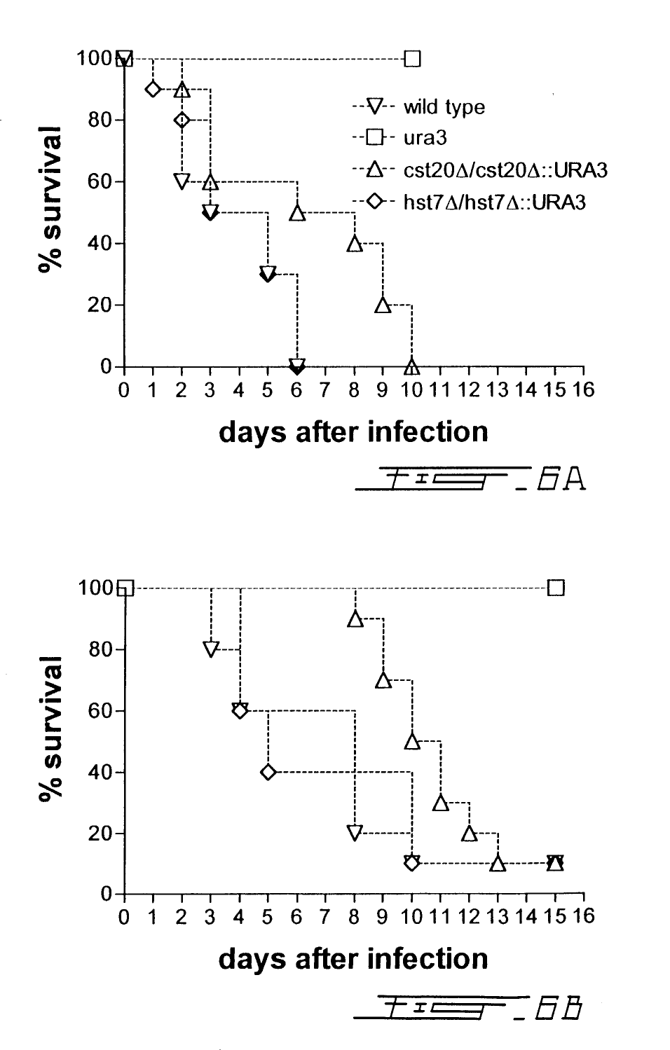

Homologous recombination was used in a multistep procedure to partially delete CST20 in a URA⁻ C. albicans strain (FIG. 4A). PCR with the divergent oligodeoxynucleotides ODH68 and ODH69 was used to partially delete the coding sequence of CST20. AhisG-URA3-hisG cassette was then inserted. The deletion was confirmed by Southern blot analyses (FIG. 4B). The genomic DNA samples digested with XhoI were from following strains: Lane #1, CAI4 (ura3/ura3 CST20/CST20); lane 2, CDH15 (ura3/ura3 CST20/cst20Δ::hisG-URA3-hisG); lane 3, CDH18 (ura3/ura3 CST20/cst20Δ::hisG); lane 4, CDH22 (ura3/ura3 cst20Δ::hisG-URA3-hisG/cst20Δ::hisG); lane 5, CDH25 (ura3/ura3 cst20Δ::hisG/cst20Δ::hisG). Northern blots showed that the CST20 transcript was absent in the corresponding homozygous deletion strains.

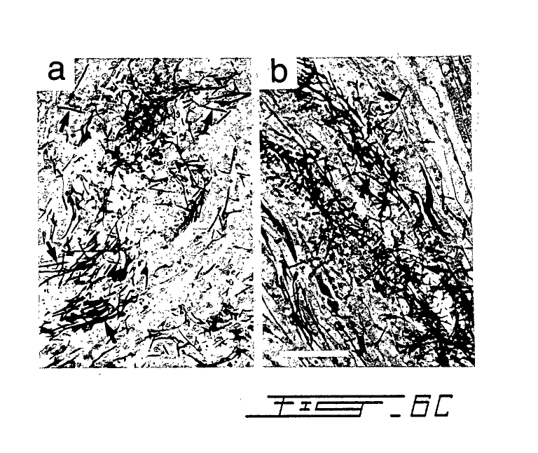

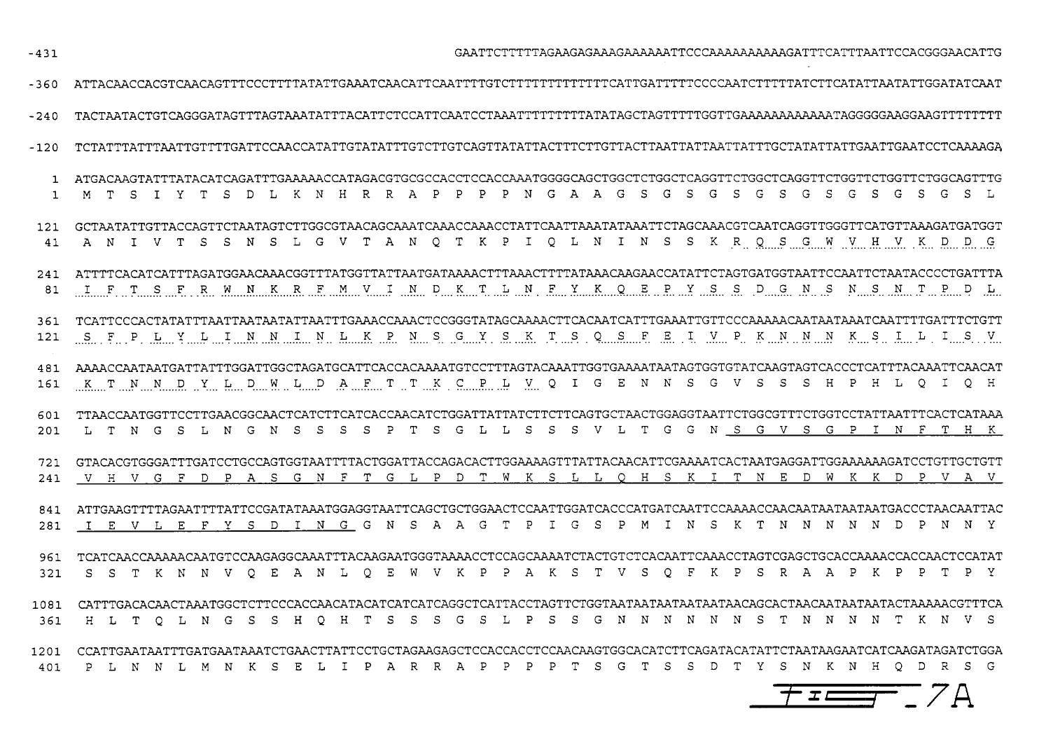

The lateral outgrowth of hyphae from colonies grown on solid “Spider” media containing mannitol or sorbitol was completely blocked by deletion of CST20 (FIG. 5B).

Mycelial formation was drastically reduced when the media contained galactose, mannose or raffinose. The mutant strains regained the ability to form hyphae when wild type CST20 was reintroduced by transformation with the CST20 expression plasmid pDH188 or reintegrated into the genome by targeted homologous recombination (FIG. 5C). The CST20 transcript was detected in these strains by Northern blot analysis.

Mutant strains formed hyphae when colonies were grown on “Spider” media containing either glucose or N-acetyl glucosamine. Normal hyphae formation was also observed on rice agar and on agar containing Lee's medium or 10% serum. The frequency of germ-tube formation in either liquid Lee's medium, 10% serum or liquid “Spider” media containing any of the sugars tested above, were also normal. These results indicate that Cst20p is not required for hyphae formation under all conditions but are involved in the lateral formation of mycelia on some solid surfaces.

Chromosomal Deletion of CaCLA4

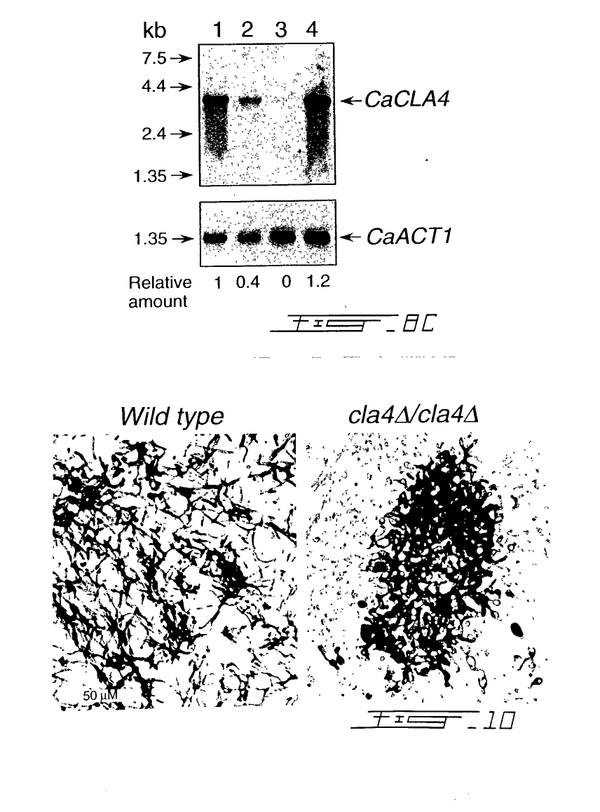

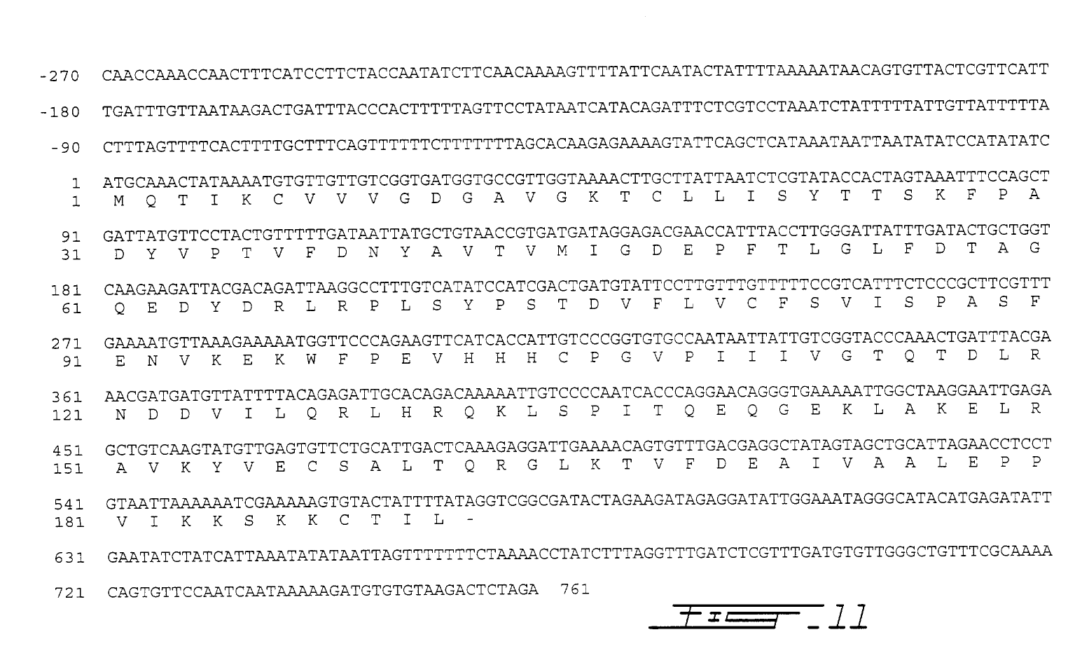

Homologous recombination was used in a multistep procedure to delete both alleles of CaCLA4 in C. albicans (FIG. 8A). FIG. 8A shows the restriction endonuclease map of CaCLA4. The coding sequence is indicated by the arrow. PCR with the divergent oligodeoxynucleotides OEL109 and OEL110 was used to delete the coding sequence of CaCLA4. A hisG-URA3-hisG cassette was then inserted and a two-step procedure was used to delete both alleles of CaCLA4 by homologous recombination. The endonuclease restriction sites are as follows: B, BamHI; Bg, BglII; E, EcoRI; H, HindIII; P, PstI; S, SacI; X, XbaI. The deletions were confirmed by Southern blot analyses (FIG. 8B). Southern blot analysis with a 1.1 kb CaCLA4 fragment from PstI-XbaI as a probe. The genomic DNA samples digested with EcoRI were from following strains: Lanes: 1, CAI4 (ura3/ura3 CaCLA4/CaCLA4); 2, CDH77 (ura3/ura3 CaCLA4/cacla4Δ::hisG-URA3-hisG); 3, CDH88 (ura3/ura3 CaCLA4/cacla4Δ::hisG); 4, CLJ1 (ura3/ura3 cacla4Δ::hisG-URA3-hisG/cacla4Δ::hisG); and 5, CLJ5 (ura3/ura3 cacla4Δ::hisG/cacla4Δ::hisG). Northern blots showed that the CaCLA4 transcript with a size of 4.1 kb was reduced to about 40% in heterozygous CaCLA4/cacla4Δ cells and was absent in homozygous cacla4Δ/cacla4Δ deletion cells (FIG. 8C). The transcript was present at about wild-type levels when the CaCLA4 gene was retransformed into the homozygous deletion cells by using an autonomously replicating plasmid carrying the CaCLA4 gene (FIG. 8C). Northern blot analysis of poly(A)⁺ RNA isolated from following strains grown in the yeast form in YPD at 30° C.: Lanes: 1, SC5314 (wild-type); 2, CDH88; 3, CLJ5 transformed with pVEC; 4, CLJ5 transformed with pVEC-CaCLA4. The blot was probed with fragments specific for CaCLA4 (upper panel) or CaACT1 (lower panel) and quantified by radioimaging. Numbers at the bottom of the figure depict the relative amounts of CaCLA4 transcript in relation to the amounts of CaACT1 transcript (mean values of two independent experiments).

It was found that viability of C. albicans cells was not affected by deleting either one or both alleles of CaCLA4. Mutant cells showed the same growth behavior as wild-type cells, independently whether the cells were grown under conditions favoring either the yeast or filamentous forms. However, deletion of both CaCLA4 alleles generated defects in cellular morphology producing a heterogeneous population of aberrantly shaped cells that were frequently multibudded and multinucleated. This phenotype indicates a defect in cytokinesis resembling the phenotype of S. cerevisiae cells deleted for CLA4 (Cvrckova, F. et al. (1995) Genes Dev. 9, 1817-1830).

Deletion of both CaCLA4 alleles caused defects in hyphal formation in all media and under all conditions that we investigated. When morphological switching was induced in liquid media by either serum, N-acetyl glucosamine, proline, pH increase, temperature shift, or Lee's medium, wild-type cells and cells deleted for only one or both alleles of CaCLA4 produced germ tubes after about 30 minutes. In wild-type cells and cells deleted for only one allele of CaCLA4, these germ tubes elongated and grew into long hyphae after prolonged incubation. Cells deleted for both alleles of CaCLA4 failed to produce hyphae, however. Instead, these cells produced multiple short protrusions giving rise to an aberrant morphology.

On solid media containing either serum, rice agar or mannitol, the normal formation of mycelia was completely suppressed by deletion of both CaCLA4 alleles. This phenotype was reversed by introducing the CaCLA4 gene on a plasmid, and deletion of only one allele had no effect.

Virulence Studies

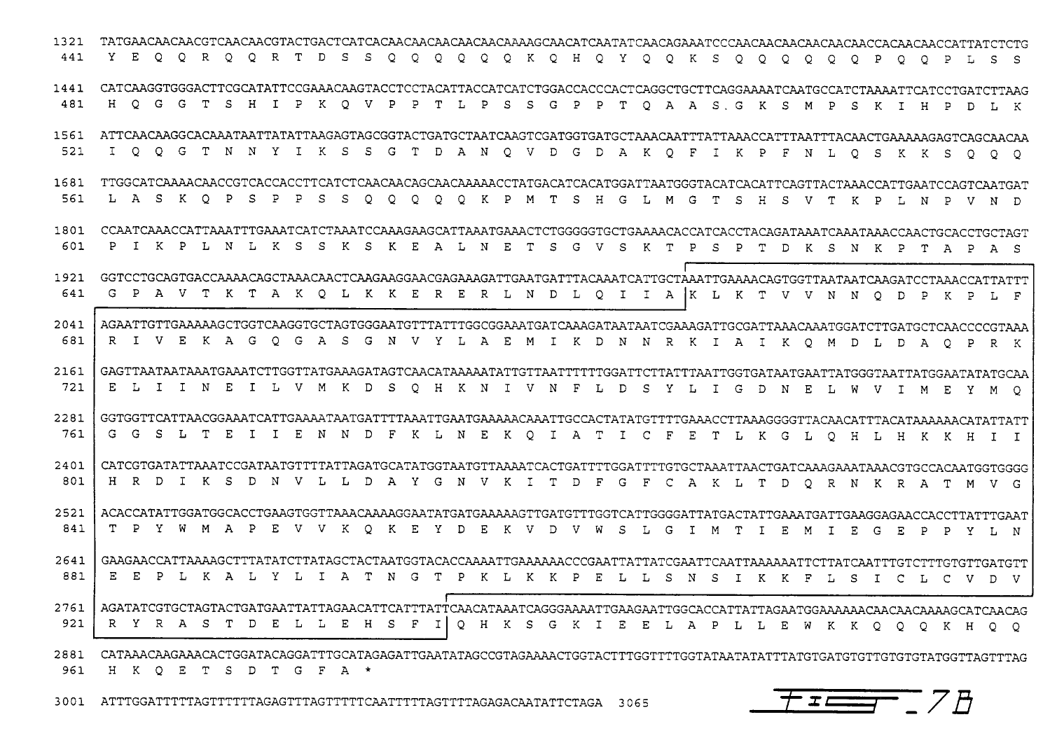

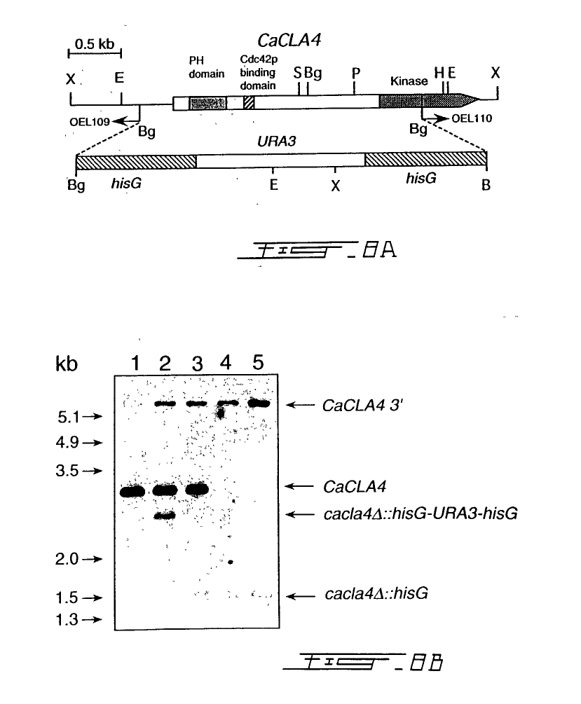

To determine the role of Cst20p for virulence, mice were injected intravenously with wild type and mutant strains and monitored for survival and for fungal invasion into kidneys. We found that the Ura⁻ strain CAI4 was not pathogenic (FIGS. 6A and B). However, infection with Ura⁺ wild type cells resulted in rapid mortality with a rate that was dependent on the dose of injected cells (1×10⁶ cells in FIG. 6A, and 1×10⁵ cells in FIG. 6B). Survival was significantly prolonged, however, in mice infected with Ura⁺ cells deleted for both alleles of CST20 (cst20Δ/cst20Δ::URA3). This effect, which was reproducible and statistically significant, was observed at high (FIG. 6A) or low (FIG. 6B) doses of infection (with P values of 0.027 and 0.001, respectively) and correlated with colony-forming units per kidney (1.5×10⁶ for wild type cells and 7×10⁵ for cst20Δ/cst20Δ::URA3 mutant cells) after 48 hours of infection with 1×10⁶ cells. These effects on virulence could be reversed by reintroducing CST20 into the strain deleted for both CST20 alleles, and were not observed in Ura⁺ cells deleted for only one CST20 allele. A histological examination revealed that cells deleted for both alleles of CST20, were able to form hyphae in infected kidneys (FIG. 6C).

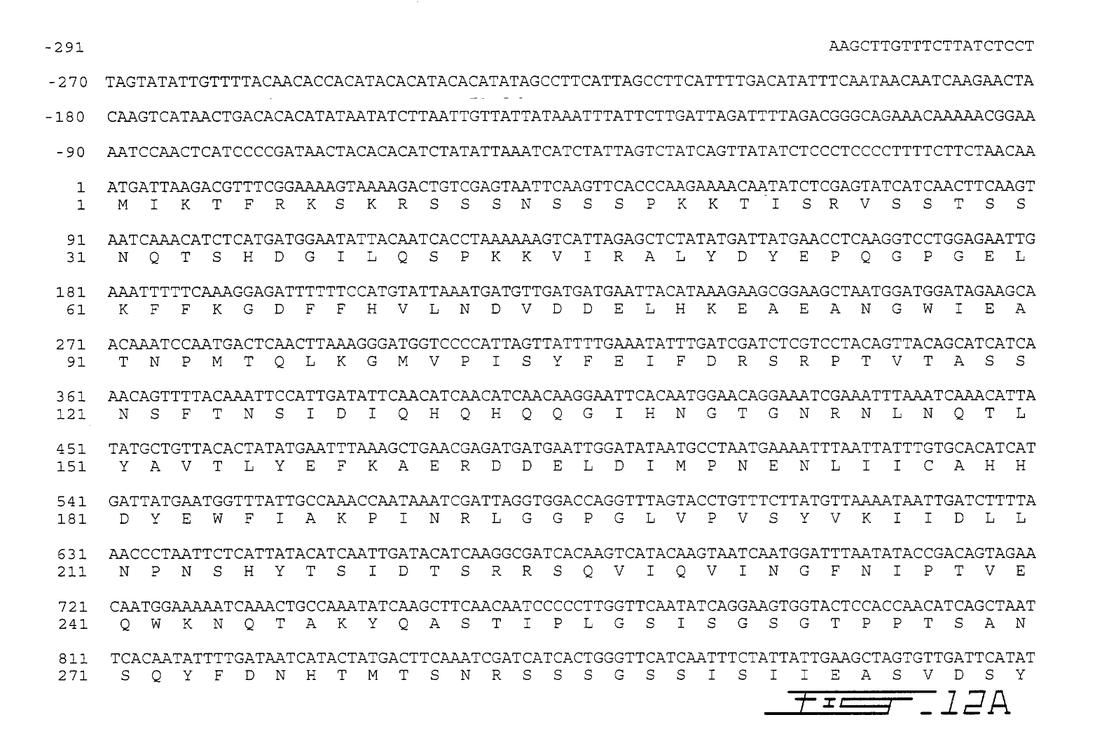

To investigate whether CaCla4p is required for virulence, mice were injected intravenously with wild-type and mutant C. albicans strains and monitored for survival and for fungal invasion into kidneys. Infections with CaCLA4 wild-type cells (strain SC5314) resulted in rapid mortality (FIG. 9). No difference in the mortality rate was observed after infection with cells deleted for only one allele of CaCLA4 (strain CDH77). All mice survived, however, after infection with cells deleted for both alleles of CaCLA4 (strain CLJ1 and CLJ5pVEC1). This effect correlated with a reduction in the amount of colony-forming units per kidney of infected animals and was reversed by transformation of the cells with a plasmid carrying the CaCLA4 gene (strain CLJ5CaCLA4) (FIG. 9). A histological examination revealed that kidneys from mice injected with either wild-type cells or cells deleted for one allele of CaCLA4 were heavily infected with C. albicans cells that produced hyphae densely penetrating the animal tissue (FIG. 10, left panel), whereas kidneys from mice injected with cells deleted for both CaCLA4 alleles contained small foci of aberrantly shaped cells that frequently carried multiple protrusions (FIG. 10, right panel). The morphologies of these cells were similar to those induced by serum under in vitro conditions. Thus, the function of CaCla4p is required for morphological switching of C. albicans under in vitro and in vivo conditions and for virulence.

Results Associated with CaCDC42

Molecular Cloning of the CaCDC42 Gene

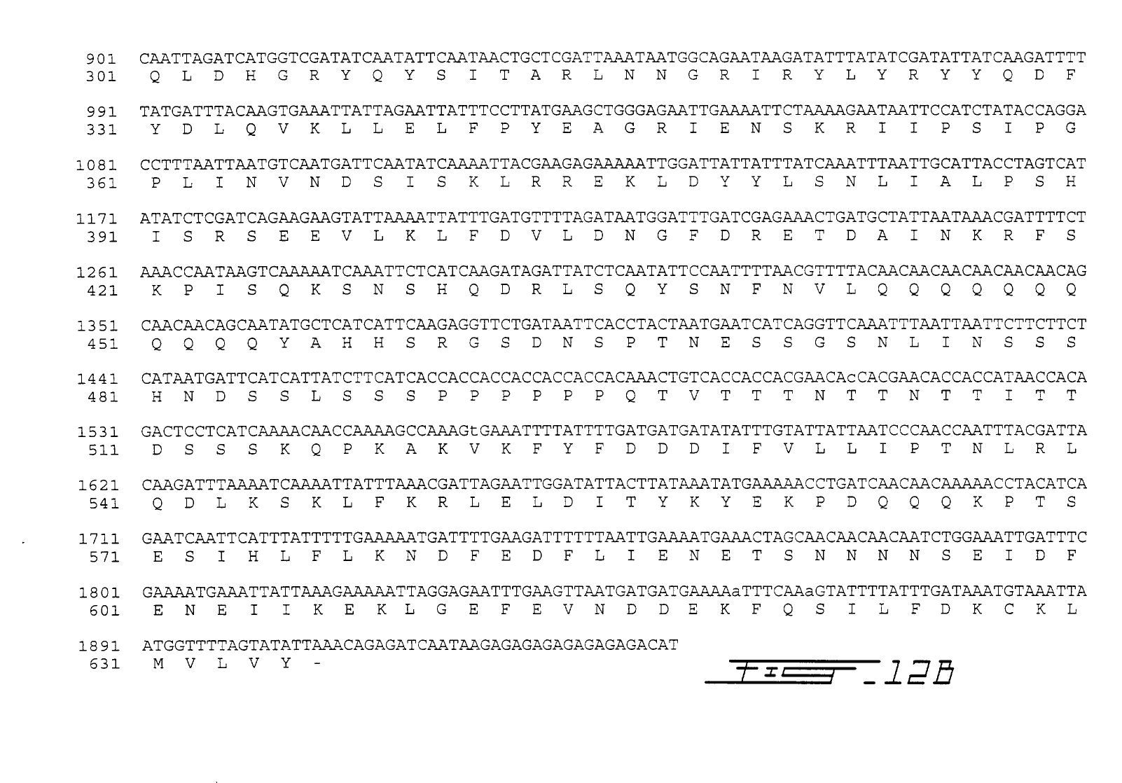

A C. albicans homolog of the CaCDC42 gene was cloned by functional complementation of the temperature-sensitive growth defect of S. cerevisiae cells carrying the cdc42-1^{ts} mutation. The growth defect was fully complemented by plasmid YEp352-CaCDC42. The open reading frame of the CaCDC42 gene is capable of encoding a protein of 191 amino acids with homology to the Rho-family of small G-proteins (FIG. 11). The highest homology is found with Cdc42p from S. cerevisiae.

CaCdc42p is Required for Vegetative Growth.

To investigate CaCDC42 function in the yeast form of C. albicans, both copies of CaCDC42 were deleted in a strain containing a copy of CaCDC42 under the control of the PCK1 promoter. This strain was subcultured in liquid media that represses PCK1 expression (2% glucose-ura) and monitored microscopically at various timepoints after subculture. By 6 hrs the majority of the culture had arrested as large, round, unbudded cells suggesting that CaCdc42p is necessary for bud formation and for polarized growth. By fluorescence activated cell sorting (FACS) analysis, the average size of CaSU96 or CASU97 after 6 hours in 2% gluc-ura was increased by 2.3 fold and 2.1 fold respectively. 4′,6′-diamino-2-phenylindole (DAPI) staining was used to determine the nuclear content of the CaCdc42p depleted cells. The majority of the arrested cells contained two nuclei, and the proportion of binucleate cells remained essentially constant upon continued inhibition of CaCdc42p function. Monitoring the cultures by FACS analysis showed the size of CaSU96 or CASU97 cells continued to increase to 3.4 fold and 4.7 fold respectively in glucose media compared to cells grown in casamino acids (CAA) after 18 hours. In addition, cellular polarity was investigated by staining cells with calcofluor to monitor chitin distribution. In contrast to wild type strains where chitin staining was primarily localized to bud sites, the CaCdc42p depleted cells were round and showed delocalized calcofluor staining.

Overexpression of Cdc42 Point Mutants in Vegetatively Grown Cells

As described in Materials and Methods, wild type CDC42 and a series of point mutant derivatives were fused to the PCK1 promoter and integrated into either strain CAI4, which contains both wild type copies of CaCDC42, or into strain CaDH85, which contains one copy of CaCDC42 disrupted. The G12V mutation has decreased intrinsic GTPase activity resulting in a mutant protein locked in an activated GTP-bound state (Ziman et al., 1991), while the D118A mutant protein does not undergo GDP to GTP exchange resulting in a protein locked in the inactive GDP-bound (Ziman et al., 1991). The C188S mutant protein cannot be isoprenylated and therefore is not localized to the membrane (Ziman et al., 1991). These cells were grown overnight in PCK1 inducing media (2% CAA-ura), stained with DAPI and examined microscopically. Overexpression of the wild type CaCdc42p in strain CaDH85 had no effect on cell proliferation or cell morphology; cells both formed colonies after overnight growth on solid medium and looked normal. In contrast, overexpression of either the hyperactive CaCdc42^{G12V} protein or the dominant negative D118A protein in strain CaDH85 blocked cell proliferation. The PCK1 mediated expression of the G12V allele generated aberrant multibudded cells while overexpression of the dominant negative D118A protein resulted in large, round cells that accumulated nuclei. The proliferation arrest and the cell morphology changes caused by overexpression of the hyperactive and dominant negative alleles require a functional CaCdc42p CAAX box. The C188S mutation rescued the unviability caused by overexpression of the G12V and D118A allele proteins, and generated morphologically normal cells (data not shown). In strain CAI4 that contains the two wild type copies of CaCDC42 in addition to the PCK1 driven gene, the phenotypic effects of the mutant proteins are similar but not as severe.

Overexpression of CaCdc42p^{G12V} in a CST20-Deleted Strain or CaCLA4-Deleted Strain

PCK1CaCDC42 (pJA28) and PCK1CaCDC42^{G12V} (pSU48) were integrated into strains doubly deleted for either CST20 (Leberer et al., 1996) or CaCLA4 (Leberer et al., 1997b) and the phenotypes were examined under PCK1 inducing or non-inducing conditions. Overexpression of CaCdc42p^{G12V} in a CaCLA4 deficient strain resulted in unviable cells that were severely elongated and multibudded; prolonged incubation lead to extensive lysis of the cells. This was not observed in the CaSTE20-deleted strain overexpressing CaCdc42p^{G12V}. A minor growth defect was observed on solid media but the cells appeared normal upon microscopic analysis. There was no significant effect of overexpressing CaCDC42 in either of these strains. The influence of the PCK1CaCDC42^{G12V} allele on cell growth was temperature dependent; room temperature expression had a more detrimental effect on proliferation than expression at 30° C., although at either temperature the CST20 deleted strain grew better than either the control or the CaCLA4 deleted strain.

CaCdc42p Involvement in Hyphal Formation

A time course experiment was done to examine the effect of depletion of CaCdc42p on hyphal formation (as described in Materials and Methods). After CaCdc42p is largely depleted (incubation for 4 hrs in glucose-ura and then hyphal formation is induced with 10% FBS for 2 hrs), the cells arrest and are large, round, and primarily binucleate. If the cells are immediately switched to 2% glucose-ura+10% serum, short germ tubes are initiated but then stop as CaCdc42p is depleted. These cells also increase in size and arrest with primarily two nuclei.

Discussion

In S. cerevisiae, Ste20p fulfills multiple functions during mating (Leberer, E. et al. (1992) EMBO J. 11, 4815-4824), pseudohyphae formation (Liu, H., Styles, C. & Fink, G. R. (1993) Science 262, 1741-1744), invasive growth (Roberts, R. L. & Fink, G. R. (1994) Genes Dev. 8, 2974-2985) and cytokinesis (Cvrckova, F. et al. (1995) Genes Dev. 9, 1817-1830). CST20 expression in S. cerevisiae fully complements these functions. Thus, Cst20p has the potential to fulfill similar functions in C. albicans.

The yeast-to-hyphal transition of C. albicans is a morphological change that can be triggered by a wide variety of factors. Carbohydrates, amino acids, salts, and serum have been described as inducers of germ tube formation, as have pH changes, temperature increases and starvation, but no single environmental factor could be defined as uniquely significant in stimulating the morphological switch. Hence C. albicans appears capable of responding to many divergent environmental signals. Disruption of both CPH1 alleles, which encode a homolog of the S. cerevisiae Ste12p transcription factor (Liu, H. et al. (1994) Science 266, 1723-1726), suppressed the lateral formation of mycelia from colonies grown on solid “Spider” medium, but did not block hyphal development in other media. We have shown that C. albicans mutant cells deleted for CST20 display a similar phenotype, and that the effect of these mutations on hyphal development is dependent on the carbon source in which the cells were grown.

These observations are consistent with the idea that several signaling pathways can trigger morphogenesis in C. albicans. Furthermore, the behavior of C. albicans mutant strains deleted for either CPH1 or CST20 indicates that these pathways might operate independently to activate hyphal development under differing environmental conditions. C. albicans encounters a variety of different microenvironments during the development of superficial and systemic infections. Hence, the existence of parallel morphogenetic signaling pathways might provide a distinct advantage to this pathogen.

The results indicate that the pathway controlled by Cst20p is not essential for virulence in a mouse model of systemic infections. It is not inconceivable that this pathway plays a role in other forms of infections, for example in the development of superficial infections of the mucosal epithelia (thrush). An as yet undefined role of Cst20p in pathogenicity outside of the Cst20 signaling pathway is suggested, however, by prolonged survival of mice infected with cst20 deleted cells. It is unlikely that this effect is caused by defects in hyphal formation since a histological examination of infected kidneys revealed that the CST20 deleted cells are not restricted in their capacity to form hyphae.

In S. cerevisiae, Cla4p plays a role in cytokinesis and shares with Ste20p an essential function for polarized growth during budding (Cvrckova, F. et al. (1995) Genes Dev. 9, 1817-1830). Cla4p binds the Rho-like small G-protein Cdc42p (Cvrckova, F. et al. (1995) Genes Dev. 9, 1817-1830), which is involved in controlling cell polarity during budding and in response to pheromone. Like Ste20p and the mammalian homolog p21-activated kinase (p65^{PAK}), Cla4p is able to phosphorylate and activate myosin-I, a mechanism that may contribute to the organization of the actin cytoskeleton.

The finding that CaCLA4 expression in S. cerevisiae completely complements the Cla4p functions suggests that CaCla4p may have similar properties in C. albicans. Thus, CaCla4p may be required for myosin-I driven polarized growth during hyphal formation in a mechanism that may involve the C. albicans homolog of Cdc42p. Our complementation assays in S. cerevisiae suggest that CaCla4p may share an essential function with Cst20p, the C. albicans homolog of Ste20p (FIGS. 6A and 6B). This notion suggests, together with our findings that null mutants of CaCLA4 are completely non-pathogenic (FIG. 10) and null mutants of CST20 are reduced in virulence (FIGS. 6A and 6B), that CaCla4p and Cst20p, and proteins such as CaCdc42p and CaBem1p interacting with these protein kinases, may be valid targets for the development of antifungal agents.

In Candida albicans the Homolog of the Cdc42p GTPase, CaCdc42p, is Required for Proper Proliferation and Cellular Morphogenesis

Construction of a strain with the only functional copy of the CaCDC42 gene under control of the regulated PCK1 promoter permitted the controlled shut-off of CaCDC42 expression. Vegetatively growing cells that were no longer expressing CaCDC42 ceased proliferation within several hours of promoter shut-off. Continued inhibition of CaCDC42 expression resulted in an accumulation of large, round, unbudded cells. These cells showed delocalized chitin staining, in contrast to the wild type cells that localized chitin to the bud necks and bud scars.

Because of the importance of the Rho family GTPases in the general regulation of polarized morphogenesis in eukaryotic cells (Tanaka and Takai, 1998; Valster et al., 2000; Chimini and Chavrier, 2000), we further investigated the involvement of CaCDC42 in the control of hyphal growth in C. albicans.

In C. albicans, specific external signals such as high temperature and serum are capable of inducing an essentially quantitative shift of yeast-like cells into hyphal forms (Brown and Gow, 1999; Whiteway, 2000). When C. albicans cells were depleted of CaCdc42p prior to triggering the yeast to hyphal switch, the cells arrested with phenotypes similar to that of the yeast form mutants, namely large, round, unbudded cells. When the yeast to hyphal switch was triggered at the same time as repression of CaCDC42 expression, the cells formed abortive germ tubes but then arrested as primarily binucleate, unbudded cells. This result suggests that the germ tube initiates before the CaCdc42p is depleted, but that in the absence of continuing CaCdc42p function the germ tube cannot be extended. Thus CaCDC42 appears essential for proper polarized growth of C. albicans cells growing under both yeast and hyphal inducing conditions.

As was also noted in the yeast cell growth situation, the phenotype of the dominant negative D118A allele was not identical to the null in hyphal conditions. The D118A mutant generates aberrant hyphal structures in the presence of serum, while the null mutant blocks hyphal formation under these conditions. It is possible that the growth conditions for stimulating hyphal formation are somewhat incompatible with PCK1 expression, and therefore the expression levels of CaCdc42p^{11A} are insufficient to block endogenous GTPase function when the cells are grown in the presence of serum. Alternatively, it may be that the target of the dominant negative protein plays a different role in yeast and hyphal growing cells.

The present work shows that CaCdc42p is required for both yeast and hyphal proliferation. In addition, this work establishes that simple overexpression of CaCDC42 has little effect on the cells, while modification of CaCdc42p activity through activating and dominant negative mutations has profound cellular effects. In the case of the hyperactive G12V allele it is evident that a primary effector is the Cst20p kinase. Thus it is likely that the activity of the CaCdc42p GTPase, rather than its expression, is the primary means through which CaCdc42p influences cellular morphogenesis.

The present invention will be more readily understood by referring to the following examples, which are given to illustrate the invention rather than to limit its scope.

### EXAMPLE I

### Screening Test for Inhibitors of CaCla4p and Cst20p

An in vitro assay can be used to identify compounds that inhibit the activity of the proteins CaCla4p and/or Cst20p. Inhibitors of these proteins are potentially useful to render pathogenic fungi avirulent.

Since CaCla4p and/or Cst20p are kinases, a standard kinase assay is suitable to identify compounds that inhibit their activity. In this example, myelin basic protein is used as a substrate. Such an assay is well-known in the art as described in (Wu et al., J. Biol. Chem., 1995). Under uninhibited conditions, the myelin basic protein is phosphorylated in the presence of a kinase (such as Cla4p and Cst20p) and ATP as a source of phosphate. This reaction is monitored by using labeled ATP. In the presence of a compound that inhibits kinase activity, the substrate will not be phosphorylated and this will be detected by an absence of labeled substrate in the reaction mixture.

Following the identification of an inhibitor of Cla4p and/or Cst20p, the effect of this inhibitor on the endogenous human protein kinase, p65^{PAK}, must be determined. This is determined also using a standard kinase assay as described above, substituting p65^{PAK} for Cla4p and/or Cst20p. For use as an anti-fungal agent, the desired inhibitor will selectively inhibit CaCla4p and Cst20p and not the endogenous p65^{PAK} of the patient being treated.

However, compounds inhibiting all three proteins, namely, CaCla4p, Cst20p and p65^{PAK}, would be useful in the treatment of inflammation.

### EXAMPLE II

### Screening Test for Inhibitors of CaCdc42p

An in vitro assay can be used to identify compounds that inhibit the activity of the protein CaCdc42p. Inhibitors of this protein are also potentially useful to render pathogenic fungi avirulent.

Since CaCdc42p is a GTPase, a standard GTPase assay is used to identify compounds that inhibit GTPase activity. Accordingly, in uninhibited conditions, CaCdc42p will hydrolyze GTP to form GDP. In the presence of an inhibitor, no hydrolysis of GTP will occur. This is monitored by using labeled GTP. Compounds found to inhibit GTP hydrolysis, and thus, to inhibit CaCdc42p, are potentially useful as anti-fungal agents.

### EXAMPLE III

### Screening Test for Inhibitors of CaBem1p

An in vitro assay can be used to identify compounds that inhibit the activity of the protein CaBem1p. Inhibitors of this protein are also potentially useful to render pathogenic fungi avirulent.

CaBem1p binds protein kinases such as CaCla4p and Cst20p. A kinase-binding assay as described in detail in Example IV would be useful to identify compounds that inhibit this interaction.

### EXAMPLE IV

Screening Test for Inhibitors of CaCla4p and CaCdc42p Interaction

In view of the indicated interaction between protein kinases in C. albicans, such as CaCla4p and Cst20p, and other proteins, such as CaCdc42p and CaBem1p, and the role these interactions are believed to play in the viability and virulence of C. albicans, inhibitors of such interactions are also potentially useful as anti-fungal agents. Accordingly, an in vitro assay to screen for such inhibitory compounds would be useful.

In this example, a kinase-regulator interaction assay can be used to determine inhibition between the protein kinase, CaCla4p or Cst20p, and proteins that it interacts with (interacting proteins), such as CaCdc42p and CaBem1p. The protein kinase is solid phase bound and the interacting proteins are in suspension free to interact with the protein kinase. A labeled antibody specific to the interacting protein is added to the reaction mixture. In uninhibited conditions, the interaction between interacting protein and protein kinase will occur. The labeled antibody will then bind to the bound interacting protein and this will be detected in the form of bound label. However, in the presence of an inhibiting compound, the interaction will not occur and this will be detected by the absence of bound label.

### EXAMPLE V

### A Two-Hybrid CaCdc42p and CaCla4p Interaction System in a Humanized S. cerevisiae Strain

This screening assay is based on the assumption that the interaction of the small G-protein CaCdc42p with its cellular targets Cst20p and CaCla4p is essential for viability of C. albicans cells. This assumption is reasonable based on work that has been performed in S. cerevisiae (Leberer E. et al. (1997) Embo J. 16, 83-97). This assay will be useful to detect inhibitors of this interaction and potential anti-fungal agents.

In this two-hybrid interaction system, the gene for green fluorescent protein is fused to the GAL1 promoter to provide a functional read out. This reporter gene will be integrated into a S. cerevisiae strain in which the STE20 and CLA4 genes have been replaced by the human homolog p65PAK. The CaCDC42 gene will be fused to the DNA binding domain of GAL4, and the CaCLA4 gene will be fused to the activation domain of GAL4. Interaction of the two proteins will cause green fluorescence, whereas inhibitors of the interaction will suppress fluorescence.

Non-specific inhibitors of the two-hybrid interaction system will be excluded by performing a parallel screen with unrelated fusion proteins known to interact. Compounds of general toxicity or inhibitors of the human homologs will also be excluded in this system because those compounds will not allow growth of the cells and therefore reduce the fluorescent readout in both parallel screens.

For use in the assay, a two-hybrid yeast strain carrying the GAL4-GFP fusion gene is constructed. This strain will be deleted for the CLA4 gene using the TRP1 marker as described (Leberer E. et al. (1997) Embo J. 16, 83-97). The STE20 gene will be replaced by the human PAK gene as described above. To replace the CDC42 gene by its human homolog, an integrating plasmid will be constructed carrying the HsCDC42 gene fused to a URA3 blaster gene and CDC42 flanking sequences. After linearization, the construct will be transformed into the PAK-containing two-hybrid strain, and integrants will be selected on -ura medium. The URA3 gene will then be looped out on FOA medium. The various gene disruptions and gene replacements will be verified by Southern blot analyses.

The two-hybrid vectors carrying the CaCDC42 gene fused to the GAL4-DNA binding domain and the CaCLA4 gene fused to the transcriptional activation domain of GAL4 will be constructed by standard procedures. To facilitate the interaction of the two proteins, site-directed mutagenesis is used to create a mutation in the CAAX-box domain of CaCDC42p to prevent isoprenylation and targeting of the fusion protein to the plasma membrane. For use in high-throughput screening, the present assay will be evaluated, optimized and adapted to achieve suitable conditions.

### EXAMPLE VI

### Detection of the Presence of C. albicans Using Probes

The sequences of either one of the genes CaCLA4, CST20, CaCDC42 and CaBEM1 may be used to derive probes for the detection of C. albicans using PCR techniques or hybridization assays.

### EXAMPLE VII

### Use of Nucleotide Sequences of CaCLA4, CST20, CaCDC42 and CaBEM1 to Identify Homologue from other Fungi

The nucleotide sequences of CaCLA4, CST20, CaCDC42 and CaBEM1 may be used to identify and clone homologues from other fungi.

### EXAMPLE VIII

### A S. cerevisiae-Based Screening System Using CaSte20p and the Pheromone Signaling Pathway as Drug Target

In this system, green fluorescent protein (GFP) under transcriptional control of a pheromone inducible promoter (FUS1) is used as a read out. The pheromone signaling pathway and thereby the reporter gene will be induced with pheromone in two different strains: firstly, in a strain in which STE20 is functionally replaced by the CST20 gene, and secondly, in a strain in which STE20 is functionally replaced by the mammalian homolog PAK. Compounds that block the induction of the reporter gene in the CaSTE20 strain but not in the PAK strain are expected to be specific inhibitors of the C. albicans kinase. In fact, CST20 gene is part of a pathway that links pheromone to gene expression. This dual assay represents a means by which compounds selective for CST20 can be identified, while eliminating those compounds which exhibit inhibitory action against the mammalian homolog PAK or compounds of general toxicity.

To conduct the foregoing assay, the FUS1 gene, including its promoter, is isolated by the polymerase chain reaction (PCR) from genomic DNA of S. cerevisiae and fused to the GFP gene from Aequoria victoria on a yeast expression plasmid. The function of the reporter gene is analyzed after transformation of a MATa yeast strain and induction with pheromone.

The STE20 gene is replaced in a supersensitive sst1 yeast strain by the human PAK gene using homologous recombination. For this purpose, an integrating plasmid is constructed carrying the PAK gene fused to URA3 blaster and STE20 flanking gene sequences. The construct is linearized and transformed into yeast, and integrants are selected on -ura medium. The URA3 gene is then looped out on FOA medium to gain back the ura3 marker. Correct integration of the PAK gene is confirmed using Southern blot analysis.

The humanized strain is then transformed with the FUS1-GFP reporter gene and analyzed for a functional signaling pathway by measuring green fluorescence after induction with pheromone. For use identifying inhibitors of C. albicans, this assay system will be evaluated, optimized and adapted to the scale for high-throughput screening.

### EXAMPLE IX

### Fluorescence Resonance Energy Transfer (FRET) as Probe for Protein-Protein Interactions

The engineering of different GFP mutants with altered fluorescence characteristics allows the use of fluorescence resonance energy transfer (FRET) to probe protein-protein interactions (Heim and Tsien (1996) Curr. Biol. 6, 178-182). The FRET phenomenon consists in a fluorescence transfer between a donor and a receptor fluorochrome. If excitation and emission wavelengths are compatible, the FRET is easily measurable. The main parameter of the reaction is the distance between donor and receptor, which must be in the range of nanometers. This is precisely the kind of values in protein-protein interactions.

A novel yeast assay system can be developed which uses FRET to measure the in vivo interaction between CaCdc42p and Cacla4p. The CaCDC42 gene will be fused to a GFP mutant that acts as donor, and the CaCLA4 gene will be fused to a mutant that acts as receptor. The yeast strain used as an expression system will be humanized as described in Example VIII. Inhibitors of the interaction are expected to reduce energy transfer, and this reduction can readily be measured spectroscopically. The interaction of unrelated proteins known to interact will be used as a reference to exclude non-specific inhibitors of the assay system. Compounds inhibiting the interaction of the human homologs or of general toxicity will be excluded by inhibition of growth and therefore reduced fluorescence in both screens.

To conduct such an assay, the CaCDC42 gene will be fused to the gene encoding the GFP^{Y66H} mutant as donor, and the CaCLA4 gene will be fused to the gene encoding the GFP^{S65T} mutant as receptor (Heim and Tsien (1996), Curr. Biol. 6, 178-182). The constructs will then be transformed into the humanized yeast strain described in Example VIII, and the FRET phenomenon will be analyzed in yeast cultures using fluorescence spectroscopy. The conditions for the assay will be worked out and optimized. The assay conditions will be adapted for use in microtiter plates for automated screening.

While the invention has been described in connection with specific embodiments thereof, it will be understood that further modifications are possible. This application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. An isolated gene encoding a protein of Candida albicans selected from the group consisting of CaCla4p (SEQ ID NO:8), Cst20p (SEQ ID NO: 6), CaCdc42p (SEQ ID NO: 10) and CaBem1p (SEQ ID NO: 12).

2. An isolated Candida albicans protein selected from the group consisting of CaCla4p (SEQ ID NO:8), Cst20p (SEQ ID NO: 6), CaCdc42p (SEQ ID NO: 10) and CaBem1p (SEQ ID NO: 12).

3. An in vitro screening assay for identifying a potential anti-fungal compound, said assay comprising the steps of:
a) combining at least one protein selected from the group consisting of CaCla4p (SEQ ID NO:8), Cst20p (SEQ ID NO: 6), CaCdc42p (SEQ ID NO: 10) and CaBem1p (SEQ ID NO: 12) with a target that interacts with said protein in a protein/target interaction;
b) adding a test compound to the protein/target mixture; and
c) measuring the protein/target interaction, wherein an interruption of the protein/target interaction indicates that said test compound is a potential inhibitor.

4. An assay as defined in claim 3, wherein said at least one protein is CaCdc42p, and the target is Cacla4p.

5. An assay as defined in claim 3, wherein said protein is CaCdc42p and the target is Cst20p.

6. An assay as defined in claim 3, wherein said protein is CaCla4p and the target is CaBem1p.

7. An assay as defined in claim 3, wherein said protein is CaBem1p and the target is Cst20p.
